SEQUENCE LISTING (9 sequences)

SEQ ID NO: 1 - protein, 41 aa, Artificial Sequence
  Human TCAP 1
QQLLSTGRVQGYDGYFVLSVEQYLELSDSANNIHFMRQSEI

SEQ ID NO: 2 - protein, 41 aa, Artificial Sequence
  Human TCAP 2
QQLLSTGRVQGYEGYYVLPVEQYPELADSSSNIQFLRQNEM

SEQ ID NO: 3 - protein, 40 aa, Artificial Sequence
  Human TCAP3
QLLSAGKVQGYDGYYVLSVEQYPELADSANNIQFLRQSEI

SEQ ID NO: 4 - protein, 41 aa, Artificial Sequence
  Human TCAP4
QQVLSTGRVQGYDGFFVISVEQYPELSDSANNIHFMRQSEM

SEQ ID NO: 5 - protein, 41 aa, Artificial Sequence
  Mouse TCAP1
QQLLGTGRVQGYDGYFVLSVEQYLELSDSANNIHFMRQSEI

SEQ ID NO: 6 - protein, 41 aa, Artificial Sequence; identical to SEQ ID NO: 2
  Mouse TCAP2

SEQ ID NO: 7 - protein, 40 aa, Artificial Sequence; identical to SEQ ID NO: 3
  Mouse TCAP3

SEQ ID NO: 8 - protein, 41 aa, Artificial Sequence
  Mouse TCAP 4
QQVLNTGRVQGYDGFFVTSVEQYPELSDSANNIHFMRQSEM

SEQ ID NO: 9 - protein, 40 aa, Artificial Sequence
  Trout TCAP3
QLLSGRKVLGYDGYYVLSIEQYPELADSANNIQFLRQSEI

(19) United States
(12) Patent Application Publication
(10) Pub. No.: US 2006/0063143 A1
(43) Pub. Date: Mar. 23, 2006

(54) Immortalized hypothalamic neuronal cell lines

(76) Inventors: Denise Belsham, Toronto (CA); David Lovejoy, Stouffville (CA)

Correspondence Address: MCCARTHY TETRAULT LLP, BOX 48, SUITE 4700,, 66WELLINGTON STREET WEST, TORONTO, ON, M5K 1E6, CA

(21) Appl. No.: 10/511,591

(22) PCT Filed: May 2, 2003
(86) PCT No.: PCT/CA03/00621

§ 371 Date: Aug. 10, 2005

Related U.S. Application Data
(60) Provisional application No. 60/376,879, filed on May 2, 2002.
(60) Provisional application No. 60/377,231, filed on May 3, 2002.

(51) Int. Cl. C12N 5/06; C12Q 1/00

(52) U.S. Cl. 435/4; 435/354

(57) ABSTRACT

The present invention is directed to a method of preparing cell lines of hypothalamic origin. The method involves infecting fetal hypothalamic cells with a retroviral vector harbouring a viral oncogene, preferably SV-40 large T antigen, followed by selection and cloning. A plurality of cell lines have been prepared which express a variety of neuronal markers. The cell lines of the present invention are useful in the development of experimental models and in the treatment of disease.

This application claims priority from U.S. application Nos. 60/376,879, filed May 2, 2002, and 60/377,231, filed May 3, 2002, which are incorporated by reference herein in their entirety.

### FIELD OF THE INVENTION

The present invention is directed to immortalized, hypothalamic neuronal cell lines to methods of making same and to methods and uses thereof.

### BACKGROUND OF THE INVENTION

The mammalian central nervous system is the most complex organ system with an estimated 10¹² different neurons that are considered biochemically and phenotypically unique (1). Neuronal cell types are typically defined by their individual patterns of neurotransmitter hormone secretion, expression of cell surface molecules and receptors, signaling properties, and morphology. The hypothalamus is a critical part of the brain that is considered by many to be the life control system in the body. It comprises a complex array of distinct neuronal phenotypes, each expressing a specific complement of neuropeptides, neurotransmitters and receptors. The neuroendocrine hypothalamus consists of a complex array of distinct neuronal phenotypes, each expressing a specific complement of neuropeptides, neurotransmitters and receptors (1). Many of our vital needs, such as those for growth, reproduction, nutrition (e.g. food and drink), sleep, and stress responses, depend on hormonal balance or homeostasis, which is controlled by both external and internal stimuli at the hypothalamic level. The hypothalamus produces a number of peptide releasing factors and neuropeptides that in turn control the activities of the pituitary and other organs in the body. Knowledge of the control mechanisms of unique peptineurgic neurons from the hypothalamus is critical before we can understand how the brain achieves its diverse central control of basic physiology. Numerous studies have been undertaken to map the afferent connections between distinct hypothalamic neurons utilizing methodology such as double-, and recently, triple-based immunocytochemistry and in-situ hybridization. (2-5). These studies are useful to generate an emerging picture of the potential cellular communication within the hypothalamus, but are not comprehensive and do not address the mechanisms involved in gene regulation and cellular signaling.

The lack of appropriate cell models is currently hindering these studies, as analysis of brain slices or whole animal experimentation yields limited, and often conflicting, mechanistic data. Non-transformed primary hypothalamic cultures are difficult to maintain, have a short life-span and represent a heterogeneous neuronal and glial cell population, usually with a minimal number of healthy peptide-secreting neurons.

The complexity of the hypothalamus, due to numerous cells harbouring unique characteristics and identities, represents a major difficulty in the direct study of the cellular biology of individual neurons or glia from this region of the brain. In particular, expression of specific neuropeptides, which characterise the identity of these unique neurons, are detected in relatively small populations of cells and, as evidence suggests, are distributed throughout the region. Thus the study of the mechanism of action of a specific neuropeptide, its gene regulation and both its original or mediated roles and contributions within the hypothalamus is limited in situ. A way to attenuate this complexity and to investigate such questions at molecular and cellular level is the establishment of clonal immortalized hypothalamic neuron- or glia-like cell lines that express the cellular markers of interest.

Historically it has proven to be difficult to establish immortalized hypothalamic cell lines, due to the lack of naturally occurring CNS tumors and the inherent difficulty of transforming or immortalizing highly differentiated neurons from primary culture (2). Cell lines from the peripheral nervous system have been established from neuroblastomas, such as the Neuro2A cell line, and pheochromocytomas, such as the PC12 cell line, however these models are not truly representative of differentiated CNS neurons. Previous infection of primary cultures of mouse hypothalamic tissue from embryonic day 14 with SV40 large T-antigen in the early 1970's produced cell lines that were not considered fully differentiated (3). On the other hand, targeted tumorigenesis in transgenic mice has been used successfully to establish cell lines in specific tissues, such as the anterior pituitary and pancreas (4-9). In an attempt to produce a suitable model to study the gonadotropin-releasing hormone (GnRH) gene, a directed tumorigenesis technique was used to develop a murine immortal cell line of GnRH-secreting hypothalamic neurons (GT1 cells e.g. GT1-7 or Gn11) (10). The cells were developed by targeting expression of the potent oncogene, SV40 T-antigen, with the regulatory region of GnRH in transgenic mice (11). This cell line represents the only hypothalamic cell model available for study and expresses a single neuropeptide, GnRH. Thus, there remains a real and unmet need for neuronal cell lines to provide valid model systems for molecular and biochemical investigations of neuronal cells.

### SUMMARY OF THE INVENTION

The present invention addresses the need for appropriate hypothalamic cell models for investigations at the cellular and molecular level.

It is an object of the invention to provide immortalized hypothalamic neuronal cell lines expressing specific markers characteristic of individual cell types and to a method for establishing same.

The present invention provides a series of clonal, immortalized hypothalamic neuronal and glial cell lines that express a viral oncogene, and specific neuronal or glial markers. At least 36 independent cell lines have been identified thus far which express unique phenotypes.

In accordance with one aspect of the invention, there is provided a method of making a hypothalamic cell line comprising:
(i) preparing a culture of embryonic hypothalamic cells;
(ii) infecting said culture with a retrovirus encoding a viral oncogene, operably linked to a promoter and a selectable marker;
(iii) isolating transfected cells from non-transfected cells to obtain a culture of immortalized hypothalamic cells;
(iv) subcloning said immortalized cells; and
(v) screening subcloned cells for expression of specific neuronal markers.

In another aspect of the invention, there is provided a cell line prepared according to the above-described method.

In one aspect, the embryonic cells are mammalian cells.

In a preferred embodiment, the embryonic cells are human.

In another preferred embodiment, the embryonic cells are murine cells harvested at day 15, 17 or 18 of gestation.

In a further preferred embodiment, the viral oncogene is large T antigen of SV-40.

According to another aspect of the invention, there is provided a cell line of hypothalamic origin expressing a specific marker.

In a preferred embodiment, at least 90%, preferably 100%, of the cells express the specific marker.

In another preferred embodiment, the cell line of the present invention is used in transplantation.

The cell lines of the present invention are useful in the development of animal models of disease and in the treatment of disease.

In another aspect of the invention, there is provided an immortalized cell line of murine hypothalmic neuronal cells comprising a gene encoding polyoma virus large T antigen operably linked to a promoter and expressing at least one marker selected from the group consisting of neuropeptide Y, gonadotropin-releasing hormone, growth-hormone releasing hormone (GHRH), TenM 1, 2, 3, 4, arginine vasopressin (AVP), thyrotropin-releasing hormone (TRH), SOCS-3, urocortin, melanocortin-concentrating hormone (MCH), orexin, dopamine transporter, corticotrophin-releasing factor (CRF), gonadotropin releasing hormone receptor, tryptophan hydroxylase, tyrosine hydroxylase, galanin, proopiomelanocortin (POMC), proglucagon, neurotensin, somatostatin, agouti-related protein, cocaine and amphetamine-regulated transcript (CART), leptin, oxytocin, corticotrophin-releasing factor receptor 1 and 2, aromatase, ghrelin, growth hormone secratogue receptor, androgen receptor, estrogen receptor α, estrogen receptor β, leptin receptor, melanocortin-concentrating hormone receptor 3 and 4, neuropeptide Y receptor Y1, neuropeptide Y receptor Y2, calcitonin receptor like receptor, glucagon-like peptide 1 receptor, glucagon-like peptide 2 receptor (Glp-2 receptor), and neurotensin receptor.

In one embodiment, the marker is Glp-2 receptor.

In another embodiment, the marker is neurotensin.

In another embodiment, the marker is proopiomelanocortin (POMC).

In another embodiment, the marker is neuropeptide Y (NPY).

In another embodiment, the marker is proglucagon.

In another embodiment, the marker is growth-hormone releasing hormone.

In another embodiment, the marker is urocortin.

In another embodiment, the marker is melanocortin-concentrating hormone.

In another embodiment, the marker is TenM 4.

In another embodiment, the marker is growth hormone secratogue receptor.

In another embodiment, the marker is ghrelin.

In a further aspect of the invention, the immortalized cell line comprises a mixed cell population.

In another aspect of the invention, the immortalized cell line is prepared by the method comprising:
(i) preparing a culture of embryonic hypothalamic cells;
(ii) infecting said culture with a retrovirus encoding a viral oncogene, operably linked to a promoter and a selectable marker;
(iii) isolating transfected cells from non-transfected cells to obtain a culture of immortalized hypothalamic cells;
(iv) subcloning said immortalized cells into sub-cloned populations;
(v) screening said subcloned populations for expression of specific neuronal markers; and
(vi) selecting and further cloning a specific population.

In an embodiment of the invention, there is provided a method of obtaining a neuropeptide comprising, culturing the cell line of the invention that is known to express said neuropeptide and isolating the expressed neuropeptide.

In one embodiment of the invention, there is provided a method for identifying a modulator of a neuropeptide comprising:
(i) providing the cell line defined above;
(ii) incubating the cell line in the presence of the candidate modulator; and
(iii) determining the biological effect of said candidate modulator,
   wherein said candidate is a modulator if it modulates the neuropeptide expression and/or activity.

In another embodiment, the invention provides a method of identifying a modulator of a neuropeptide wherein said effect of said candidate modulator can be determined by one of the following methods:
(a) monitoring effects on neuropeptide expression;
(b) incubating the said cell line with a substrate of a neuropeptide and monitoring the effect on said substrate, such as by monitoring metabolites of said substrate;
(c) binding assays; or
(d) proteomic profiling in the presence and absence of the said candidate modulator.

In a further aspect of the invention, there is provided an immortalized cell line of murine hypothalmic neuronal cells that is responsive to a teneurin C-terminal-associated peptide [TCAP].

In an embodiment of the invention, the teneurin C-terminal-associated peptide is selected from the group consisting of SEQ ID NOs 1-9.

In a further embodiment of the invention, the teneurin C-terminal-associated peptide is murine TCAP-1 or TCAP-3.

In another embodiment of the invention, the cell line is selected from the group consisting of N-7, N-22, N-29 and N-38.

### BRIEF DESCRIPTION OF THE DRAWINGS

Preferred embodiments of the invention are described with respect to the drawings, wherein:

FIG. 1 is a phase contrast microscopic view of the mixed neuronal cell population, derived from day e17 fetal mouse hypothalamus, expressing SV40 large T-antigen (T-Ag);

FIG. 2 illustrates RT-PCR of the mixed cultures of immortalized hypothalamic cultures (e15 and e17) derived using SV40 large T-antigen primers (T-Ag, 433 bp fragment). Left panel: neuron-specific enolase primers (NSE, 391 bp fragment), and Right panel: neurofilament (NF). GT1-7 cell and hypothalmic RNA were used as positive controls, whereas reactions without reverse transcriptase (RT-) were also included.

FIG. 3A illustrates RT-PCR of cDNA from a number of subcloned hypothalamic cell lines initially using SV40 large T-antigen primers (T-Ag, 433 bp fragment);

FIG. 3B illustrates RT-PCR of the subcloned cell lines using neuron-specific enolase primers (NSE, 391 bp fragment);

FIG. 4 illustrates cell line screening for CART, GHRH, AGRP, and leptin receptor;

FIG. 5 illustrates cell line screening for NPY, galanin, proglucagon, POMC, and tyrosine hydroxylase;

FIG. 6 illustrates an agarose gel stained with ethidium bromide of RT-PCR fragments for neurotensin, and mouse TenM neuropeptides 1, 2, and 4 (New neuropeptide 1, 2 and 4);

FIG. 7 illustrates cell line screening for glp-2 receptor (G2R), and GnRH;

FIG. 8 illustrates cell line screening for CRF;

FIG. 9 demonstrates positive clones for GFAP;

FIG. 10 illustrates cell line screening for arginine vasopressin;

FIG. 11 illustrates the expression of ERα and ERβ;

FIG. 12 illustrates four phase contrast micrographs of NPY-17 neuronal cells (derived from day e17 fetal mouse hypothalamus) stained with 0.01% methylene blue;

FIG. 13 illustrates RT-PCR of NPY-17, GT1-7 and hypothalamic cDNA using SV40 large T-antigen primers (T-Ag, 433 bp fragment), neuron-specific enolase primers (NSE, 391 bp fragment), neuropeptide Y primers (NPY, 282 bp), and glial fibrillary acidic protein primers (GFAP, 621 bp);

FIG. 14 demonstrates specific staining with a mouse polyclonal NPY antibody (Peninsula Laboratories) in the NPY-17 neurons;

FIG. 15 illustrates the neurosecretory response of NPY-17 (N 38) cells. The effect of the addition of KCl (60 mM) on intracellular calcium levels ([Ca2+]i) for the cells pictured is shown. Images of cells shown in the two panels on the right were obtained as per Example 5. Shown is a representative experiment from 6 experiments, n=116 cells in total (FIG. 15, left panel);

FIG. 16 illustrates the effect of sequential deletions of the NPY 5′ regulatory region on expression in NPY-17 neurons;

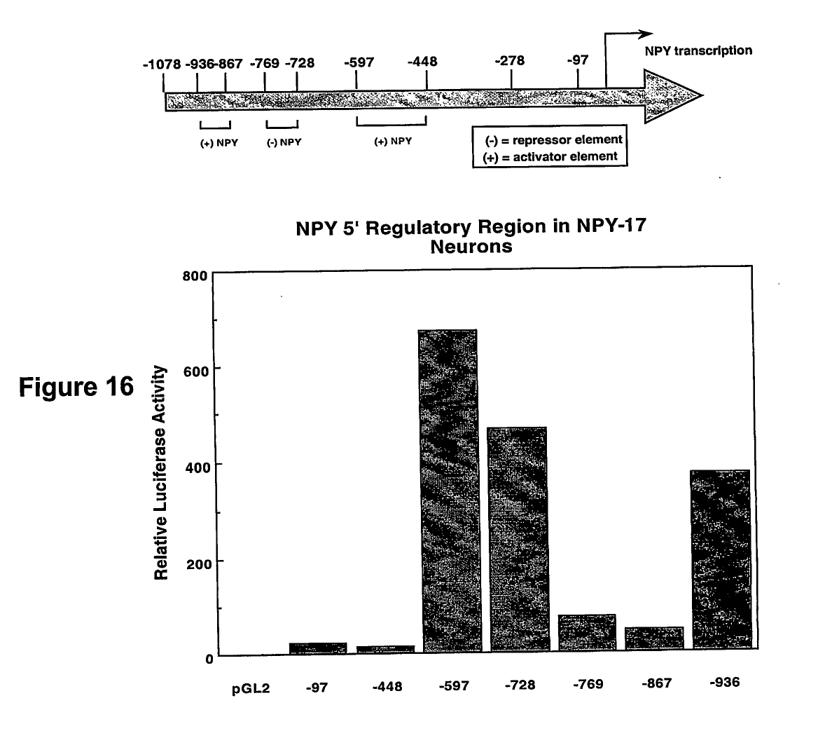

FIG. 17 illustrates the expression of both estrogen receptor α (ERα) and estrogen receptor β (ERβ), and AR in the NPY-17 cells;

FIG. 18A illustrates the functional cAMP response of the immortalized subclones to TCAP peptide stimulation; and

FIG. 18B illustrates the response to glucagon-like peptide 2.

FIG. 19 illustrates that immortalized, clonal cell lines express neuronal cell markers, but exhibit unique cellular morphologies and neuroendocrine markers. (A) Representative phase contrast micrographs of clonal cell lines, N-1, N-4, N-6, N-20, N-36 and N-38. (B) Electron micrographs of N-38 exhibiting dense core material (i, ii) and cell contact regions (iii, iv). (C) RT-PCR of NSE, T-Ag, and GFAP, or (D) NPY, AgRP, and Ob-Rb in N-38 cells. GT1-7 cell and hypothalamic RNA are used as positive controls, whereas reactions without reverse transcriptase (RT-) were also included, as indicated.

FIG. 20 illustrates a comparison of human, murine and rainbow trout TCAP peptide sequences [SEQ ID Nos 1-9].

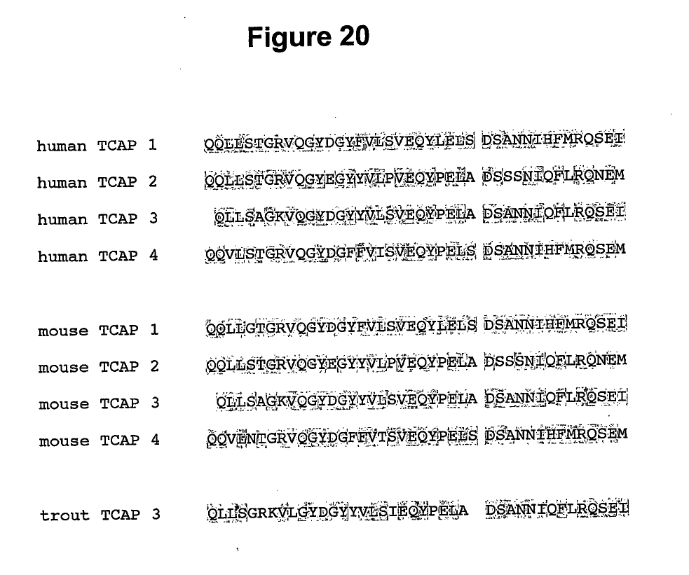

### DETAILED DESCRIPTION

The present invention provides a series of clonal, immortalized hypothalamic neuronal cell lines that express the oncogene Simian virus (SV 40) large T-antigen and specific neuronal markers and a method for producing same. A method for the generation of a number of clonal, immortalized hypothalamic neuronal and glial cell lines is also provided. Several immortalized cells lines have been expanded and characterized.

“Immortalized cell line” as used herein means a cell line that can replicate and be maintained indefinitely in in vitro cultures under conditions that promote growth, preferably at least over a period of a year or years.

“Cell line” as used herein is a population or mixture of cells of common origin growing together after several passages in vitro. By growing together in the same medium and culture conditions, the cells of the cell line share the characteristics of generally similar growth rates, temperature, gas phase, nutritional and surface requirements. The presence of cells in the cell line expressing certain substances, for example Neuropeptide Y (NPY) can be ascertained, provided a sufficient proportion, if not all, of cells in the line are present to produce a measurable quantity of the substance. An enriched cell line is one in which cells having a certain trait e.g. expression of NPY, are present in greater proportion after one or more subculture steps than the original cell line. Preferably the cell line is derived from one, two or three originating cells. The cell line can become more homogenous with successive passages and selection for specific traits. Clonal cells are those which are descended from a single cell. A cloned cell culture is a cell culture derived from a single cell.

A SV40 Large T Antigen (SV-40 LTA) oncogene is intended to encompass any nucleotide sequence which encodes a protein having the function of polyoma (or SV-40) LTa and which is capable of being expressed in the host cell in a quantity measurable by a known assay for LTa, such as immunochemical staining.

A selectable marker is a genetic determinant which makes it possible to provide culture conditions which favour the growth of cells possessing the marker, compared to cells which do not. An antibiotic resistance gene is an example of a selectable marker.

The following abbreviations will have their standard scientific abbreviations: T-Ag, Large T-antigen; NSE, neuron-specific enolase; GFAP, glial fibrillary acidic protein; SNTX, syntaxin; ER, estrogen receptor; AR, androgen receptor; LepR, leptin receptor b; Glp-2R (also G2R), glucagon-like peptide 2 receptor; SOCS-3, suppressor of cytokine signaling 3; NPY, neuropeptide Y; AGRP, agoutirelated peptide; POMC, proopiomelanocortin; CART, cocaine and amphetamine regulated transcript; MCH, melanin-concentrating hormone; Ucn, urocortin; NT, neurotensin; Gal, galanin; Orx, orexin; DAT, dopamine transporter; CRFR, corticotrophin-releasing factor receptor; proGlu, proglucagon; GHRH, growth hormone-releasing hormone; GnRH, gonadotropin-releasing hormone; GnRHR, gonadotropin-releasing hormone receptor; CRF, corticotropin-releasing factor; TRH, thyroid-releasing hormone; AVP, arginine vasopressin; OXY, oxytocin; Arom, aromatase; TPH, tryptophan hydroxylase; TH, tyrosine hydroxylase; TenM-1 (also New-1); TenM-2 (also New-2); TenM-3 (also New-3); and TenM-4 (also New-4), Teneurins 1-4; GHS-R, growth hormone secratogue receptor; Lep, leptin; SOM, somatostatin; NTR, neurotensin receptor; MC3R, melanocortin receptor-3; MC4R, melanocortin receptor-4; NPY-Y1, NPY receptor Y1; NPY-Y2, NPY receptor Y2; CRLR, calcitonin receptor like receptor; nd, not done; na, not done; w, weak expression.

The present invention utilizes embryonic cells. For the purpose of illustration herein, primary cell cultures from embryonic (e) day 18, 17 and 15 fetal mouse hypothalamus were used representing a period of recognized neurogenesis. After preparing a single cell suspension, the cells are plated and are preferably allowed to grow for about twenty-four hours.

The primary cell cultures were then infected with a retrovirus which harbours the sequence for SV-40 Large T antigen (T-Ag) and neomycin resistance. SV-40 (T-Ag) is known to cause tumor transformation. A preferred virus according to the present invention is a replication defective, recombinant murine virus which is harvested from psitex cells.

The retrovirus infected cells are incubated in the presence of geneticin (G418) and resistant colonies are selected and expanded. After several passages, cells were subcloned and during each expansion step, a part of the culture was stored in liquid nitrogen.

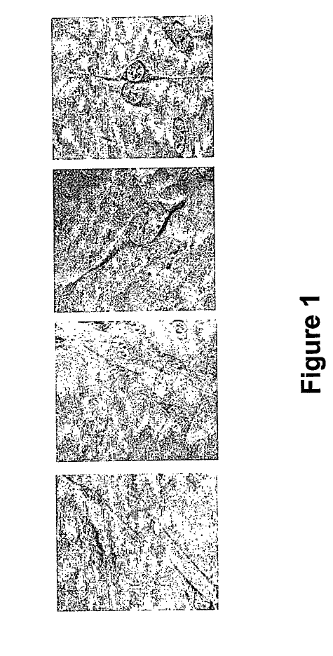

A phase contrast microscopic view of the mixed neuronal cell population, derived from day e17 fetal mouse hypothalamus, expressing SV40 large T-antigen is shown in FIG. 1. Cells were cultured on a cover slide in 1×DMEM with 10% FBS (Gibco). Cells shown are at passage 3. Cells are stained with 0.01% methylene blue for 20 minutes, after fixation with paraformaldehyde. The magnification is 400×.

The mixed cell populations which comprise immortalized cells of more than one phenotype are considered to be neuron-like due to the expression of neuron-specific enolase (NSE), a specific neuronal marker, and neurofilament, but not glial fibrillary acidic protein (GFAP).

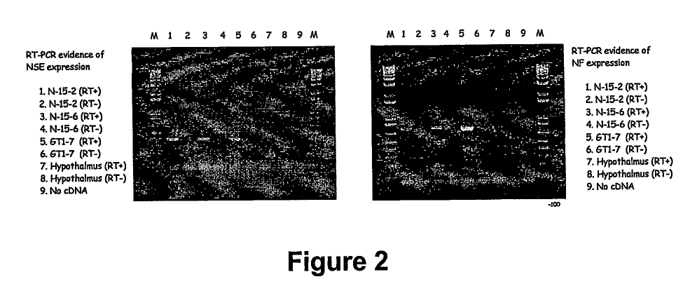

FIG. 2 illustrates the results of RT-PCR of the mixed cultures of immortalized hypothalamic cultures using SV40 large T-antigen primers (T-Ag, 433 bp fragment), neuron-specific enolase primers (NSE, 391 bp fragment), and neurofilament (NF). The control is RNA without the reverse transcriptase enzyme (RT-). PCR fragments were excised, subcloned, and sequenced to confirm identity. As a further control, the RNA used in all RT reactions was pre-treated with DNasel.

Further expansion of a cell line was performed only after the expression of large T-Ag, neuron-specific enolase and lack of glial fibrillary acidic protein was confirmed either through reverse transcriptase polymerase chain reaction (RT-PCR) or Northern blotting.

Positive cell lines were further subcloned through successive dilutions. The clones were expanded and an aliquot was frozen in liquid nitrogen. RNA was extracted from each clone and used to make cDNA. The clonal cell line was then analyzed for expression of a large number of markers indicative of a unique neuronal phenotype. A correlation of cell line names between those used in the figures and in Table 3 is listed in Table 1. A summary of the markers and cell lines screened are listed in Table 3. A list of the primers used for the screening of each marker is listed in Table 2.

The above described methodology generates a large library of potential clones of interest. Preliminary experiments yielded approximately 36 cell lines with a clonal (single) cell population or a cell population of at most 2-3 cells, that were produced through serial dilutions of the mixed populations of immortalized hypothalamic cells into 96 well tissue culture plates. Hypothalamic cells representing a mixed population were frozen in liquid nitrogen. These immortalized hypothalamic cell populations represent a virtually unlimited resource of unique neuronal cell phenotypes and can be screened for any peptide, neurotransmitter, or receptor of interest. The present invention prevails a mixed population of immortalized hypothalamic cells comprising a large T-Ag and derived by the method of the present invention.

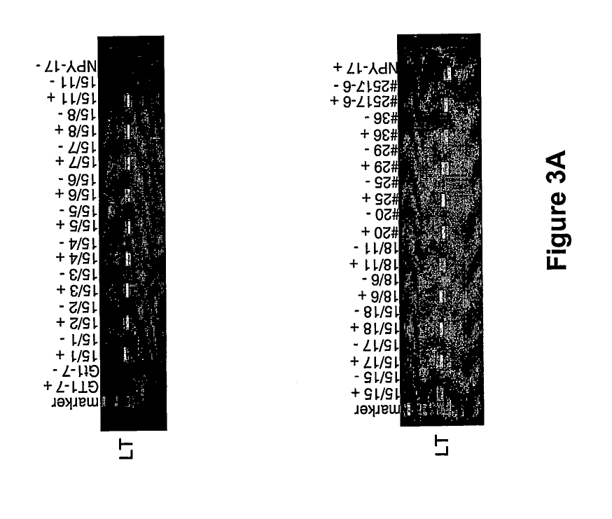

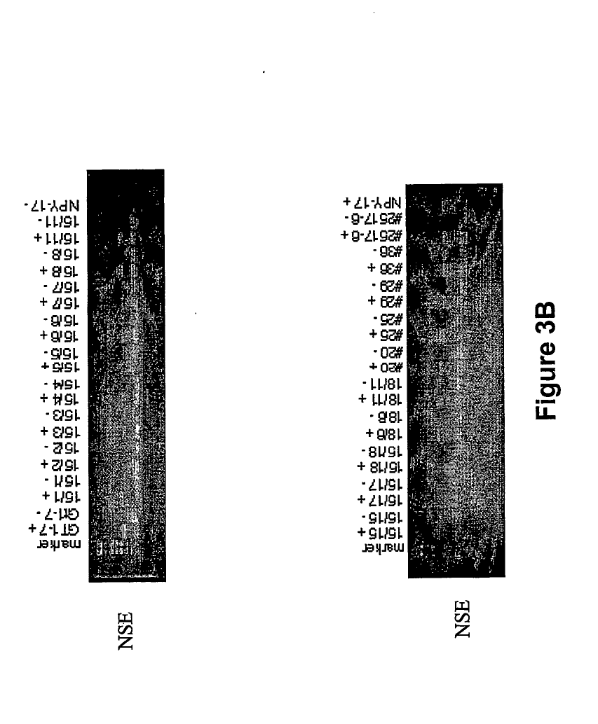

FIGS. 3A and 3B illustrate the results of RT-PCR of cDNA from a number of subcloned hypothalamic cell lines initially using SV40 large T-antigen primers (T-Ag, 433 bp fragment) (see FIG. 3A), and neuron-specific enolase primers (NSE, 391 bp fragment) (see FIG. 3B).

The cell lines were then screened for selected gene expression by RT-PCR, followed by Southern blotting in which, the blot was probed with an internal sequence to the flanking RT-PCR primers. Table 2 indicates the primers that were used. The control is RNA without the reverse transcriptase enzyme (RT-). PCR fragments were excised, subcloned, and sequenced to confirm identity. As a further control, the RNA used in all RT reactions was pre-treated with DNasel. Hypothalamic RNA and GT1-7 GnRH neuronal RNA was used as a control in the screening.

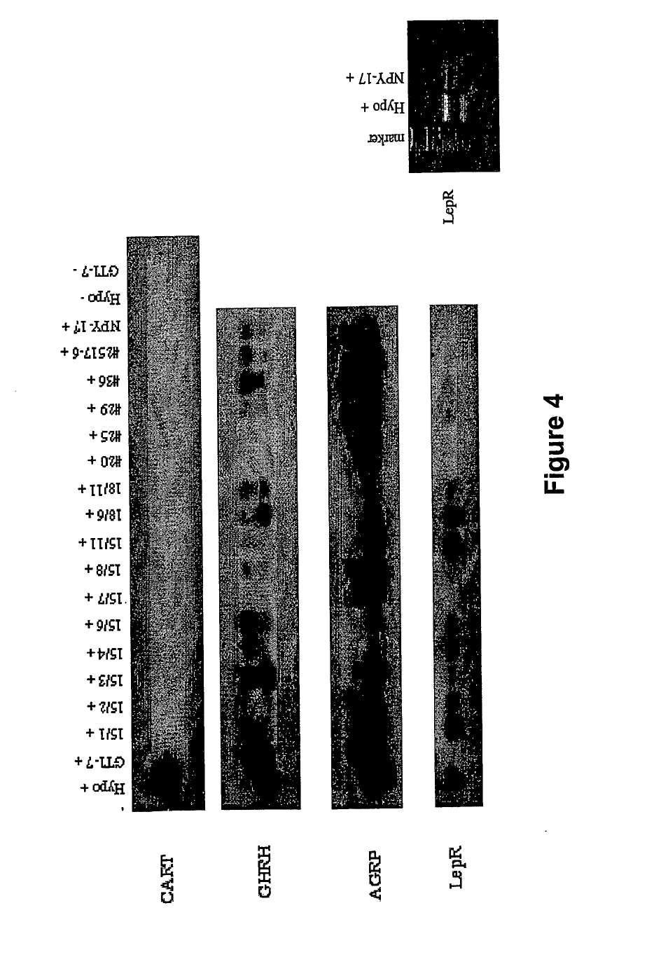

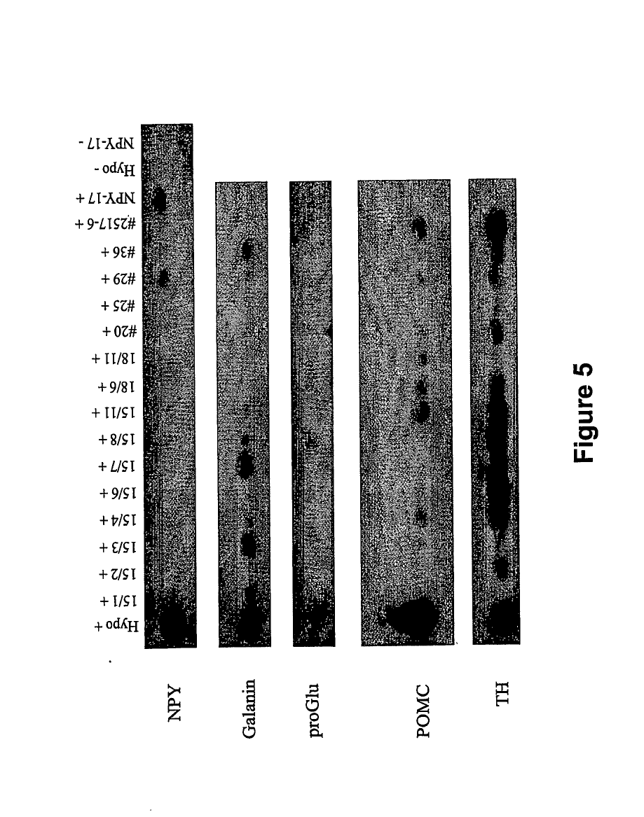

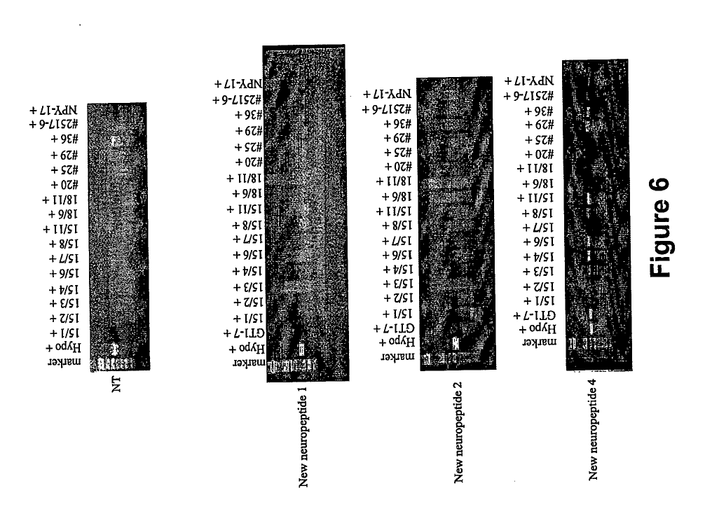

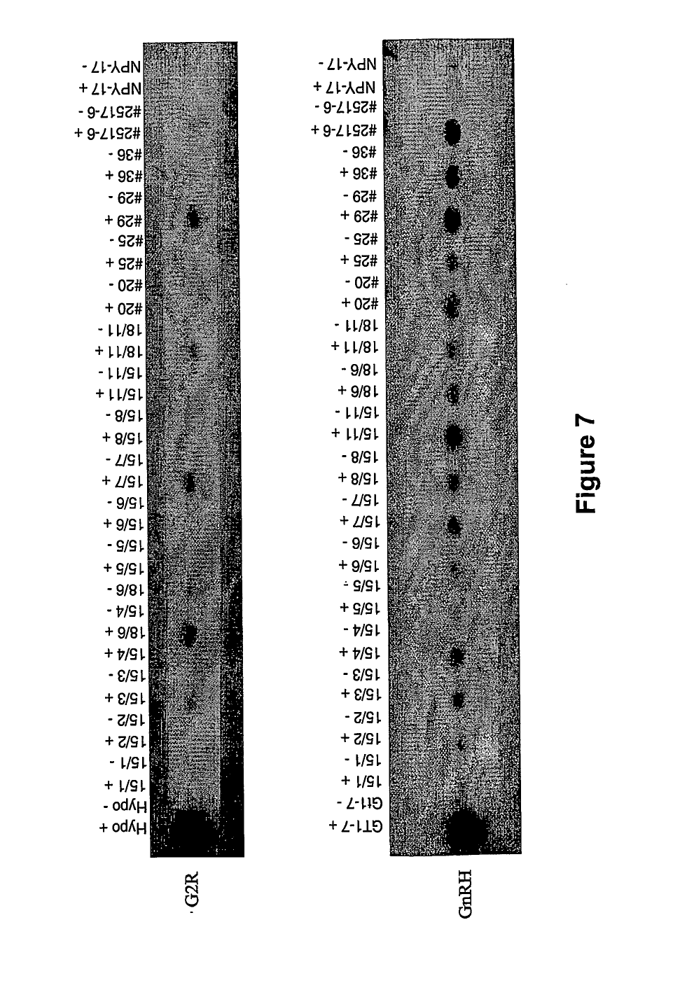

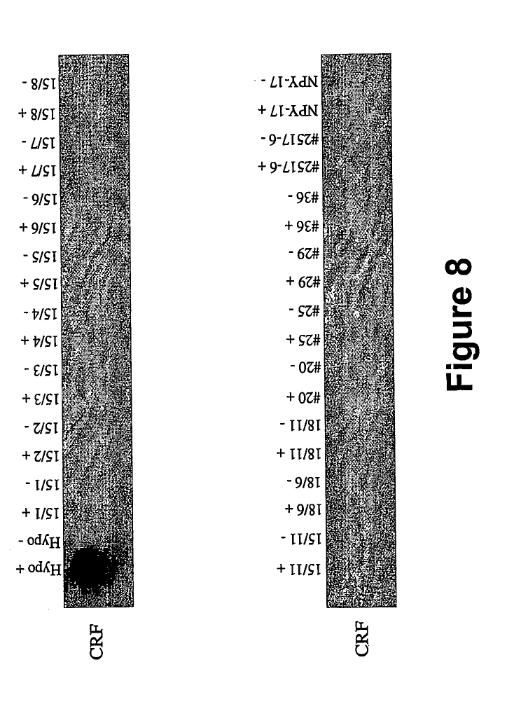

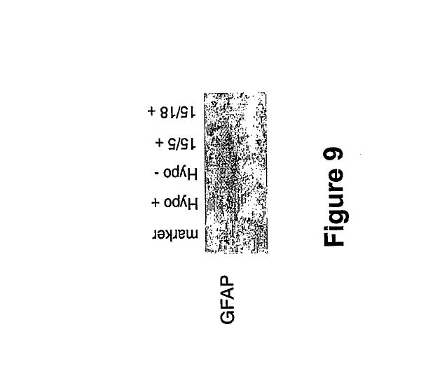

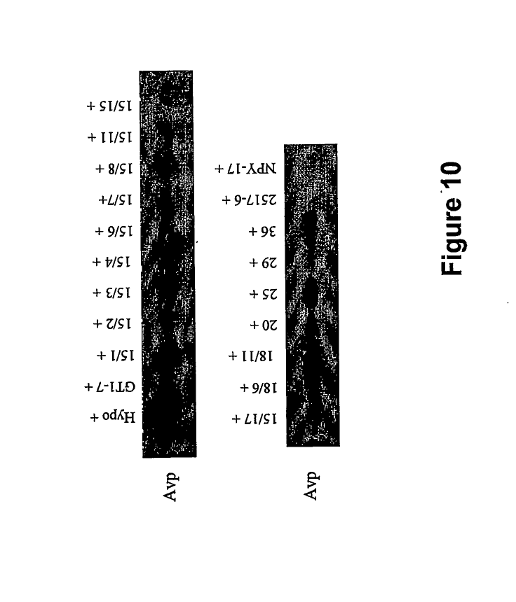

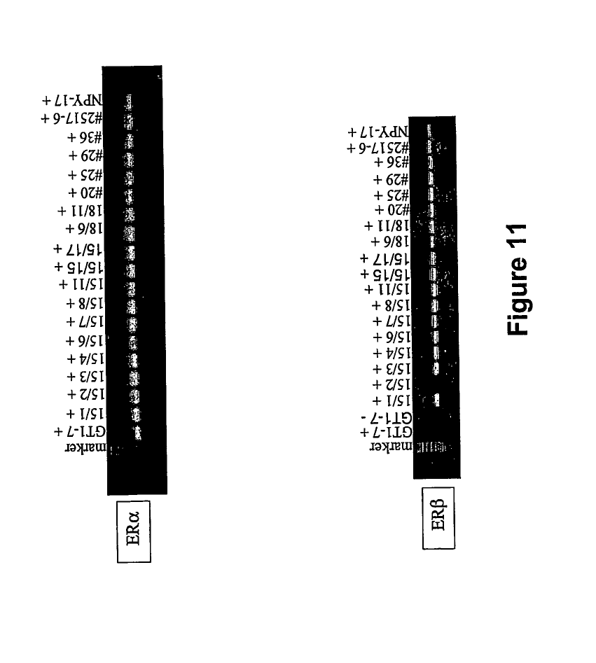

FIGS. 4 to 11 illustrate the results of cell line screening for specific markers. FIG. 4 illustrates CART, GHRH, AGRP, and leptin receptor screening. FIG. 5 illustrates NPY, galanin, proglucagon, POMC, and tyrosine hydroxylase screening. FIG. 6 is an agarose gel stained with ethidium bromide of RT-PCR fragments for neurotensin, TenM neuropeptides 1, 2, and 4. FIG. 7 illustrates screening for glp-2 receptor and GnRH and FIG. 8 illustrates the results of screening for CRF. Positive clones for GFAP are demonstrated in FIG. 9. FIG. 10 illustrates the results of cell line screening for arginine vasopressin. FIG. 11 illustrates the presence of PCR fragments corresponding to expression of both ERα and ERβ.

Once it was determined that a cell line expresses a gene of interest, another 2 rounds of subcloning was done to be sure that the cell population was indeed clonal and pure. These cells were then grown and further analysed to confirm the phenotype of the cell line. Each individual cell line was screened for an extensive list of markers. Table 2 provides a list of the neuropeptides and receptors for which the cell lines were screened. These markers are described below.

Cell lines that express these markers can be used as a source of said peptides. They can also be used in methods for identifying modulators of said compound activity or substances that may be used in the treatment of the peptide associated conditions. For instance, the cell culture of the cell lines can be incubated with a candidate modulator in order to determine the effect on the respective marker expression and/or activity using methodologies known in the art or discussed herein with respect to NPY cells and TCAP responsive cell lines.

Neuron-specific enolase (NSE) is the most abundant form of the glycolytic enzyme enolase found in adult neurons and is thought to serve as a growth factor in neurons. NSE is useful in studying neuronal differentiation and is, therefore, a valuable tool for visualizing the entire nervous and neuroendocrine systems. Serum levels of NSE have been associated with such disease states as Alzheimer's, Huntingdon Chorea, neuroblastoma, head trauma, neuroendocrine malignancies and small cell carcinomas of the lung.

Glial fibrillary acidic protein (GFAP) is selectively located in astrocytes and represents the major constituent of astrocytic intermediate filaments. In adults, GFAP levels increase as a result of the proliferation of astrocytes that occurs in response to a variety of physical, chemical and etiological insults, including Alzheimer's disease, epilepsy and multiple sclerosis.

Estrogen receptors (ERα) are transcriptional factors with a DNA binding domain and ligand binding domain and have been shown to be expressed and transcriptionally active in a number of tissues including ovary, testis, prostate and brain. The estrogen receptor has been classified into two distinct isoforms, alpha and beta. ERα is known to interact in a ligand dependent and independent manner with several known coactivators including Receptor-Interacting Protein 140 (RIP140/ERAP140), ERAP160, and Steroid Receptor Coactivator-1 (SRC-1) to enhance transcriptional activity of target genes.

Estrogen receptors (ERβ) are transcriptional factors with a DNA binding domain and ligand binding domain and have been shown to be expressed and transcriptionally active in a number of tissues including ovary, testis, prostate and brain. ERβ has been shown to be a high affinity estrogen binding protein capable of initiating transcription of genes under the control of estrogen response elements (EREs).

Leptin receptor-b (LepR) is a class 1 cytokine receptor that is expressed in high levels in the hypothalamus. Mutations in LepR have been found to cause obesity in mice and this receptor appears to be critical for the weight-reducing effects of leptin, a cytokine which targets various cells in the body.

Pro-opiomelanocortin (POMC) is a prohormone that acts as an important mediator in the regulation of feeding behavior, insulin levels and, ultimately, body weight. Bioactive peptides derived from POMC are generated in neurons of the hypothalamus and act as endogenous ligands for the melanocortin-4 receptor (MC4R), a key molecule underlying appetite control and energy homeostasis. It was determined that POMC is expressed in cell lines N-4, N-11, N-19, N-20, N-22, N-29 and N-37, and weak expression was detected in cell lines N-3 and N-8 (see Table 3). These cell lines can be used in developing diagnostic and therapeutic treatments for disorders arising from dysregulation of feeding behaviour, insulin levels, and body weight.

Galanin (Gal) is a neuropeptide that has been found to influence several physiological processes such as cognition and memory, the release of various neurotransmitters and hormones (e.g. acetylcholine, noradrenaline, glutamate, dopamine, insulin, growth hormone, prolactin), motility of the digestive tract, nociception, feeding, and sexual behavior.

Agouti-Related Protein (AGP) is a potent antagonist of the melanocortin-3 receptor (MC3R) and melanocortin-4 receptor (MC4R) and is an integral component in the metabolic processes that regulate feeding behavior and body weight.

Cocaine and amphetamine regulated transcript (CART) is a recently discovered hypothalamic peptide with a potent appetite suppressing activity. In the central nervous system CART is highly expressed in many hypothalamic nuclei, some of which are involved in regulating feeding behaviour.

Neuropeptide Y (NPY) is the most abundant neuropeptide in the brain. It is a member of a family of proteins that include pancreatic polypeptide, peptide YY and seminalplasmin. In addition to its function in feeding behavior, several other physiologic roles have been assigned to NPY, including involvement in circadian rhythms, sexual function, anxiety responses and vascular resistance. It was determined that NPY is expressed in cell lines N-29, N-38, N-25/2, N-29/4, N-40, N-41, N-42, N-43, N-44, N-45, N-46, and N-47, and weak expression was detected in cell lines N-1, N-11, N-36 N-25/3 and N-29/1 (see Table 3). These cell lines can be used in developing diagnostic and therapeutic treatments for circadian rhythm, sexual function, anxiety and vascular resistance disorders.

Growth hormone-releasing hormone (GHRH) is a mixture of two peptides, one containing 40 amino acids, the other 44. GHRH stimulates cells in the anterior lobe of the pituitary to secrete growth hormone (GH). It was determined that GHRH is expressed in cell lines N-3, N-6, N-19, N-22, N-29, N-36, N-37 and N-38, and weak expression was detected in cell lines N-1, N-2, N-8, N-11 and N-25 (see Table 3). These cell lines can be used in developing diagnostic and therapeutic treatments for disorders including growth hormone-related disorders.

Corticotropin-releasing factor (CRF) has been hypothesized to be involved in the pathophysiology of anxiety, depression, cognitive and feeding disorders. Two distinct CRF receptor subtypes, CRFR1 and CRFR2, are thought to mediate CRF actions in the central nervous system.

Urocortin (Ucn) is a peptide that binds and activates transfected type-1 corticotropin-releasing factor (CRF) receptors and is more potent than CRF at binding and activating type-2 CRF receptors. Indications are that urocortin is an endogenous ligand for the type-2 CRF receptor, which is hypothesized to be involved in the pathophysiology of anxiety, depression, cognitive and feeding disorders. It was determined that Ucn is expressed in cell lines N-1, N-6, N-11, N-20, N-25, N-36, N-39, N-20/1, N-20/2, N-25/2, N25/3, N-29/1, N-29/2, N-29/3, N-29/4, N-36/1, N-36/2, N-40, N-41, N-42, N-43, N-46 and N-47, and weak expression was detected in cell lines N-3, N-, N-25/4, N-44, and N-45 (see Table 3). These cell lines can be used in developing diagnostic and therapeutic treatments for anxiety, depression, cognitive and feeding disorders.

Melanin-concentrating hormone (MCH) is a cyclic neuropeptide that regulates a variety of functions in mammalian brain, in particular feeding behavior. MCH is thought to influence feeding and energy balance by acting downstream of leptin and the melanocortin system. It was determined that MCH is expressed in cell lines N-3, N-4, N-7, N-29, N-38, N-39, N-20/1, N-20/2, N-25/2, N25/3, N-29/1, N-29/2, N-29/3, N-29/4, N-36/1, N-36/2, N-40, N-41, N-42, N-43, N-44, N-45, N-46 and N-47, and weak expression was detected in cell lines N-36, N-37 and N-25-4 (see Table 3). These cell lines can be used in developing diagnostic and therapeutic treatments for feeding behaviour-related disorders.

The proglucagon gene (proGLU) encodes several hormones that are important in human physiology. Proglucagon-derived peptides are involved in a wide variety of both peripheral as well as central functions, such as glucose homeostasis, gastric emptying, insulin secretion and the regulation of food intake. It was determined that proGLU is expressed in cell lines N-8, N-37, N-20/2, N-40, and N-41, and weak expression was detected in cell lines N-6, N-22, N-29 and N-39 (see Table 3). These cell lines can be used in developing diagnostic and therapeutic treatments for disorders relating to glucose homeostasis, gastric emptying, insulin secretion and the regulation of food intake.

Neurotensin (NT) is an endogenous peptide that is involved with memory function, specifically Alzheimer's disease. This peptide may also be involved in the pathophysiology of Parkinson's disease and schizophrenia. It was determined that NT is expressed in cell lines N-36, N-39, N-20/1, N-20/2, N-25/2, N-29/1, N-29/2, N-29/3, N-29/4, N-36/1, N-36/2, N-40, N-41, N-42, N-43, N-44, N-45, N-46 and N-47 (see Table 3). These cell lines can be used in developing diagnostic and therapeutic treatments for Alzheimer's disease, Parkinson's disease and schizophrenia.

Oxytocin (OXT) is a neurohypophyseal hormone that has a wide range of behavioral effects outside its classic peripheral endocrine functions. OXT involvement in adaptive central nervous system processes has been demonstrated as an inhibitory, amnestic action on learning and memory in different paradigms.

Arginine vasopressin hormone (AVP) (also known as anti-diuretic hormone, ADH) is important in the regulation of the water permeability of renal collecting tubules and the ascending loop of Henle. It is also a vasoconstrictor and is thought to play a role in arterial pressure maintenance during blood loss.

Tryptophan hydroxylase (TPH) is the rate-limiting enzyme in the biosynthesis of serotonin and an important component of melatonin biosynthesis. Serotonin functions mainly as a neurotransmitter, whereas melatonin is the principal hormone secreted by the pineal gland. The TPH gene has recently been associated with behavioural disorders such as manic depression and aggression.

Tyrosine hydroxylase (TH) is the rate-limiting enzyme in the biosynthesis of catecholamines, an example being dopamine. The enzyme has been intensively studied in relation to both its physiological function in the brain and brain disorders. The study of this enzyme has led to the understanding of many diseases such as Parkinson's disease, stress and emotional disorders.

SOCS-3 is an inhibitor of cytokine receptor signaling that blocks signals from the brain that normally shut down appetite when the body's fat cells have had their fill.

Androgen receptors (AR) have been found in a variety of tissues, including reproductive organs, central nervous system and skeletal muscle.

Orexins (Orx) are a family of hypothalamic neuropeptides selectively expressed in the hypothalamus that are thought to play a role in feeding behaviour.

The dopamine transporter (DAT) is the site of presynaptic reuptake of dopamine, an event that terminates its synaptic activity.

Corticotrophin-releasing factor receptor-1 (CFFR1) is thought to mediate CRF actions in the CNS. Genetic deletion of the CRFR1 receptor can lead to impairments in anxiety-like and cognitive behaviors, supporting a critical role for this receptor in anxiety and cognitive biological processes.

Corticotrophin-releasing factor receptor 2 (CRFR2) is thought to mediate CRF actions in the CNS.

Aromatase is responsible for converting testosterone into estrogens.

Gonadotropin releasing hormone receptor (GnRH Receptor) is the receptor for gonadotropin releasing hormone, a neuropeptide that is involved in regulating reproduction.

Gonadotropin releasing hormone (GnRH) is a neuropeptide that is involved in regulating reproduction. The release of GnRH from the hypothalamus regulates the production of gonadotropins in the pituitary and these gonadotropins are responsible for gonadal development and growth in vertebrates.

Leptin is a protein hormone with important effects in regulating body weight, metabolism and reproductive function. The protein is approximately ˜16 kDa in mass and encoded by the obese (ob) gene.

Ghrelin is an endogenous ligand for the growth hormone seretagogue receptor (GHS-Rs), which regulates pituitary growth hormone (GH) secretion. It is associated with obesity and has shown to be involved in the regulation of food intake and body weight. It was determined that ghrelin is expressed in cell lines N-1, N-7, N-8, N-22, N-37, N-38, N-39, N-20/1, N-20/2, N-25/2, N-29/1, N-29/2, N-29/3, N-29/4, N-36/1, N-36/2, N-40, N-41, N-42, N-43, N-44, N-45, N-46 and N-47 (see Table 3). These cell lines can be used in developing diagnostic and therapeutic treatments for obesity and disorders involving the regulation of food intake and body weight.

Growth hormone secratogue receptor (GHS-R) is a G-protein-coupled receptor that is expressed in the pituitary gland and in several areas of the brain including the hypothalamus. It is also known as the Ghrelin receptor. It was determined that GHS-R is expressed in cell lines N-39, N-29/1, N-29/2, N-29/3, N-44 and N-46 (see Table 3). These cell lines can be used in developing diagnostic and therapeutic treatments.

Neurofilament protein (NF) is a marker of neurons, mainly axons.

Somatostatin (SOM) is a hormone that inhibits the secretion of growth hormone. Somatostatin and its synthetic analogs are used clinically to treat a variety of neoplasms. It is also used in to treat giantism and acromegaly, due to its ability to inhibit growth hormone secretion.

Estrogen receptor-associated protein 140 (ERAP-140) is a protein that has been found to interact with the estrogen receptor in a hormone dependant manner.

TAU represents a family of microtubule-associated proteins that are thought to regulate different properties of neuronal (and possibly also glial) microtubules, especially their stability and orientation. Because the spreading of intraneuronal deposits of an altered form of this protein correlates with the severity of Alzheimer symptoms, it is thought to be implicated in the pathogenesis of Alzheimer's disease.

Neurotensin receptor (NTR) is a G-protein coupled receptor for neurotensin, which is an endogenous neurotransmitter that influences distinct central and peripheral physiological functions in mammals.

Melanocortin receptor-3 (MC3R) is a receptor for melanocortin hormone, which is involved in the regulation of satiety and energy homeostasis.

Melanocortin receptor-4 (MC4R) is a receptor for melanocortin hormone, which is involved in the regulation of satiety and energy homeostasis.

NPY Receptor Y1 (NPY-Y1) is a receptor for NPY that is involved in mediating stimulatory effects on food intake.

NPY Receptor Y2 (NPY-Y2) is a receptor for NPY that is involved in mediating stimulatory effects on food intake.

Calcitonin Receptor Like Receptor (CRLR) is a receptor for Calcitonin-gene-related peptide (CGRP), a peptide with many actions ranging from vasodilation to inhibition of some of the effects of insulin on metabolism. It has 55% homology with the calcitonin receptor.

Glucagon-like peptide 2 receptor (Glp-2R or G2R) is a member of a subfamily of G protein-coupled receptors. Glp-2R is expressed on neural cells and its expression has been found in the cerebellum, medulla, amygdala, hippocampus, dentate gyrus, pons, cerebral cortex, pituitary gland, and hypothalamus. Glp-2R is thought to have a role in taste aversion and nausea, and its signaling has been shown to enhance cell survival and inhibit apoptosis. It was determined that Glp-2R is expressed in cell lines N-7, N-19, N-22, and N-29 and weakly expressed in cell line N-3 (see Table 3). These cell lines can be used in developing diagnostic and therapeutic treatments.

The teneurins are the vertebrate homologues of an enigmatic family of proteins (Ten-m) originally discovered in Drosophila (33-35). The highest levels of the ten-m genes occur in the central nervous system where the protein occurs preferentially on the surface of axons (35,36). Mutations of the Drosophila ten-m gene result in embryonic lethality (34,35). Four such teneurin paralogous genes exist in vertebrates and encode approximately a 2800-amino acid Type II transmembrane protein where the carboxy terminus of the protein is displayed on the extracellular face of the cell (32). Teneurin-4 in mammalian cells is upregulated by the transcription factor GADD153/CHOP (37), a transcription factor that is induced by several types of cellular stress or conditions triggering endoplasmic reticulum (ER) stress. Overexpression of teneurin-2 into the mouse neuroblastoma cells (Nb2a) can increase the amount of neurite outgrowth and enlarge the growth cones. Family members include TenM 1, TenM 2, TenM 3 and TenM 4. It was determined that TenM 4 (New-4) is expressed in cell lines N-1, N-2, N-3, N-4, N-6, N-8, N-11, N-19, N-29, N-36, and N-37 (see Table 3). These cell lines can be used in developing diagnostic and therapeutic treatments.

The screening generally included: Southern blotting with an internal probe to the gene of interest; cloning and sequencing of the PCR product to ensure that the DNA fragment is truly the gene of interest; and Northern analysis to determine the expression levels of the gene of interest. Functional studies, depending upon the gene expression profile, were also performed to assess the status of each cell line.

As summarized in Tables 1-3, a number of individual cell lines with expression of a number of neuropeptides, enzymatic markers, and receptor molecules have been found. RT-PCR results, followed by Southern blot and sequencing confirmation, indicates that at least one cell line expressing each of: neuropeptide Y, gonadotropin-releasing hormone, tyrosine hydroxylase, galanin, proopiomelanocortin (POMC), proglucagon, neurotensin, somatostatin, urocortin, growth hormone-releasing hormone, melanocortin-concentrating hormone and agouti-related protein has been characterized. It is clearly apparent that the initial library of mixed population of immortalized cells contains many other potential clones which have not yet been analyzed due to the sheer volume generated by the method. However, the mixed population of immortalized cells is useful for studying the interneuronal communication of cells present in the hypothalamus and for screening the effects of various substances and conditions on said mixed population of cells. These cell lines can be used in the methods described herein for the more specific cell lines on microarray analysis and through proteomic profiling and in developing diagnostic and therapeutic treatments for various neuropathological disorders. Several of the neuropeptide-expressing cell lines which were analysed, did have receptors for androgen and estrogen, leptin, melanocortin-concentrating hormone receptor 3 and 4, neuropeptide Y. glucagon-like peptide 1, glucagon-like peptide 2, and neurotensin.

NPY-17 (N-38) Cell Line

One particular subcloned cell line, NPY-17, derived at e17, synthesizes NPY and expresses neuronal, but not glial, cell markers and the receptors for a number of neurotransmitters and neuromodulators thought to be involved in the control of the NPY neuron. Neuropeptide Y is abundantly expressed in the central nervous system and is highly conserved across species (13). It has a broad range of physiological activities. The most recognized functions include the regulation of endocrine function, circadian rhythms, and satiety, but it has also been implicated in psychiatric disorders, stress response and as a cardiovascular regulator or vasoconstrictor (13). Central administration of NPY stimulates feeding and repeated doses results in an increase in body weight (14). Hypothalamic NPY and GnRH neurons display many common characteristics, as do other neurosecretory cells. The NPY gene encodes a prepro-NPY precursor, NPY is stored in secretory granules, and it is secreted in a pulsatile manner, at a similar frequency to GnRH (15, 16). The NPY gene 5′ regulatory region has been partially defined using transient transfection analysis in PC12 cells, a pheochromocytoma cell line that can differentiate into neurons -upon treatment with nerve growth factor (NGF), or LA-N-5 cells, a neuroblastoma cell line (17-20). The PC12 cell line is certainly not representative of a hypothalamic NPY neuron as the tumor originated in the adrenal gland and only expresses NPY after the cells are treated with NGF to promote neuronal differentiation. Nevertheless much of the research regarding NPY gene expression has been performed with this cell line. It is known from bilateral neural transection experiments and antisense data that the NPY neurons responsible for the reproductive and orexigenic effects of NPY lie within defined regions of the hypothalamus (21-23), which clearly are not represented by any of the tumor-derived cell lines used for the NPY studies. Further, since differential NPY gene expression has been proposed to be region and cell specific, we are poised to produce what would appear to be more representative data with the NPY-17 hypothalamic cell line. Interestingly, regulation of the gene by forskolin, a PKA activator, and phorbol ester, a PKC activator, has been found to be similar but through unique mechanisms in the two cell lines used for NPY studies, PC12 and LA-N-5 cells (24), indicating that there is cell specific regulation of the NPY gene.

This is the first report of the generation of a clonal, hypothalamic, immortalized NPY-producing neuronal cell line. The immortalization protocol of the present invention produced a number of different cell models of clonal hypothalamic neurons and glial cells. An NPY-synthesizing clone (NPY-17) was first selected for further characterization, due to the important role that NPY plays in the control of reproductive physiology.

In spite of the fact that NPY itself has such an extensive repertoire of physiological functions, relatively little research has been done with regards to the molecular mechanisms involved in the regulation of NPY transcription. Further, as previously mentioned, much of the work that has been done involved heterologous or tumor cell lines, with transfection of the NPY promoter sequences, or in brain slices/primary culture, which contain a mixed population of neurons. This is due to the fact that no suitable model of the hypothalamic NPY neurons was available for these studies. With the development of the NPY-17 cell line, it is possible to approach these studies in a clonal population of NPY-expressing cells.

The NPY-17 cells are stable in culture, with a doubling time of 1-2 days. They can be passaged extensively, maintaining expression of SV40 T-antigen. Morphologically, they appear to possess a neuronal phenotype. They have clearly defined perikarya and neurites. Some of the neurites appear as short dendritic-like processes, while others form lengthy processes. The cells are fairly large and globular, and appear to have a large nucleus, when compared to the GT1-7 neuron which are more elongated.

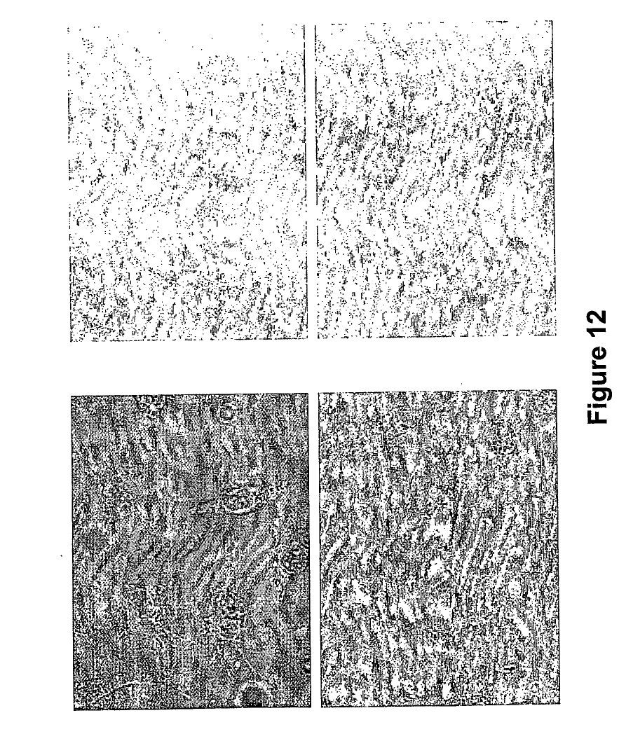

FIG. 12 is a phase contrast micrograph of NPY-17 neuronal cells (derived from day e17 fetal mouse hypothalamus). Cells were cultured on a cover slide in 1×DMEM with 10% FBS (Gibco). Cells shown are at passage 5 after purification. NPY-17 cells are stained with 0.01% methylene blue for 20 minutes, after fixation with paraformaldehyde. The magnification is 400×.

Preliminary characterization of this cell line, NPY-17, indicates that it expresses only neuronal cell markers, such as neuron-specific enolase and the 68 kDa neurofilament protein, but not glial fibrillary acidic protein, normally found in astrocytes.

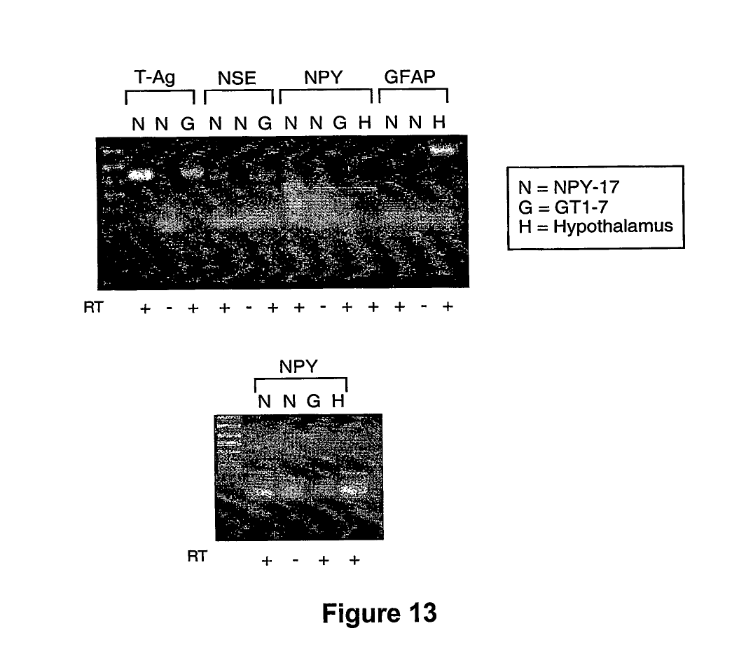

FIG. 13 illustrates RT-PCR of NPY-17, GT1-7 and hypothalamic cDNA using SV40 large T-antigen primers (T-Ag, 433 bp fragment), neuron-specific enolase primers (NSE, 391 bp fragment), neuropeptide Y primers (NPY, 282 bp), and glial fibrillary acidic protein primers (GFAP, 621 bp). Control is NPY-17 cDNA without the reverse transcriptase enzyme (RT-). PCR fragments were excised, subcloned, and sequenced to confirm identity. As a further control, the RNA used in all RT reactions was pre-treated with DNasel.

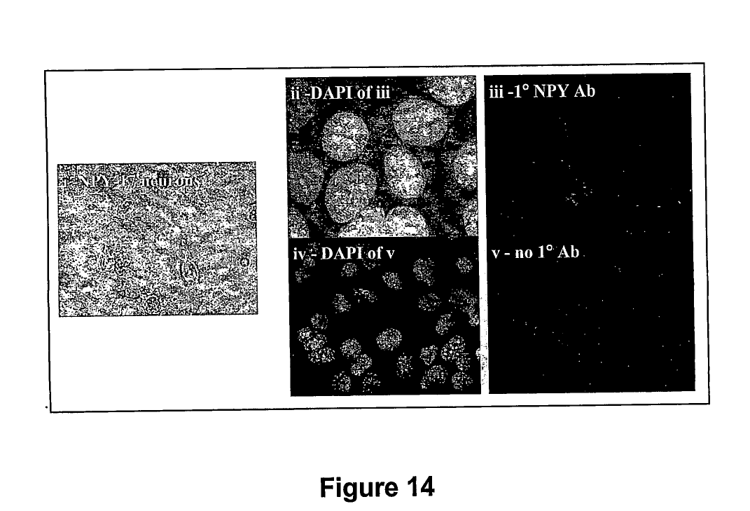

The NPY-17 cells express NPY, as detected by RT-PCR and immunocytochemistry. As shown in FIG. 14, immunocytochemistry with a mouse polyclonal NPY antibody (Peninsula Laboratories) displays specific staining in the NPY-17 neurons (i). NPY-17 cells were plated on poly-L-lysine coated coverslips, washed with PBST, and preincubated with goat serum. Cells were then incubated with the NPY primary (1°) antibody (Peninsula Labs) for 24 hours (iii), followed by secondary antibody. Immunoreactivity was visualized with a Texas Red fluorescent tag. Cell nuclei are visualized with DAPI fluorescence (ii). Parallel staining was done with no incubation with 1° NPY antibody (iv and v).

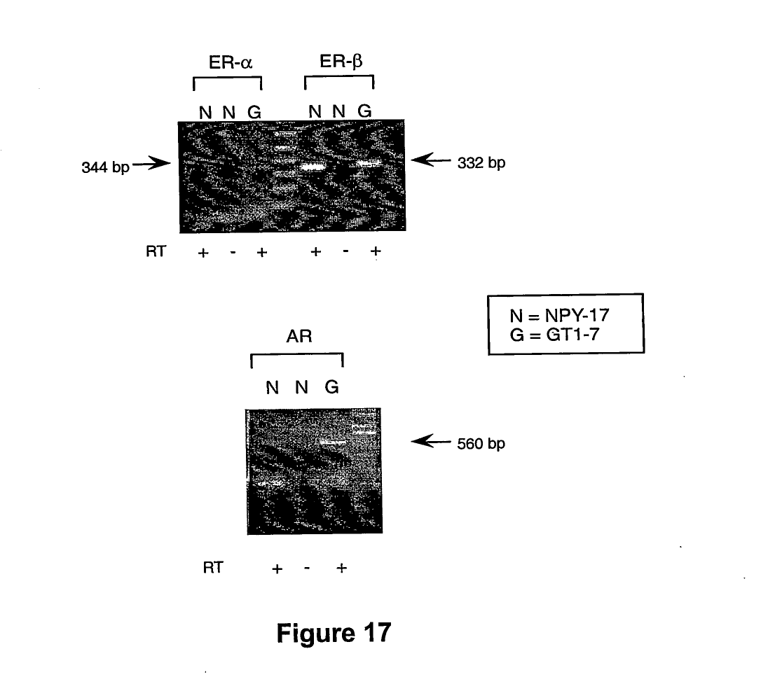

Using RT-PCR, it was found that the NPY neurons express estrogen receptors, α and β, and androgen receptors. FIG. 17A illustrates RT-PCR of NPY-17 and GT1-7 cDNA using ERα primers (344 bp fragment) and internal ERβ primers (332 bp fragment). The ERβ fragment was too weak to visualize with the ERβ external primers, so the external ERβ PCR product was reamplified with internal primers to ERβ. The control is NPY-17 cDNA without the reverse transcriptase enzyme (RT-). PCR fragments were excised, subcloned, and sequenced to confirm identity. FIG. 17B illustrates RT-PCR of NPY-17 and GT1-7 cDNA using AR primers (560 bp fragment). The control is NPY-17 cDNA without the reverse transcriptase enzyme (RT-). As a further control, the RNA used in all RT reactions was pre-treated with DNasel.

The NPY-17 cells also express leptin receptors and have been studied for leptin responsiveness using both gene array analysis and proteomic analysis. NPY-17 cells were treated with 10-7 M leptin (recombinant mouse, Bachem Scientific) for 20 hours. Cells were washed with PBS and harvested through centrifugation. Nuclear extracts were prepared and exposed to mass spectrometry analysis. Preliminary analysis of the data indicates that approximately 250 proteins are altered by leptin treatment. The analysis is not considered quantitative, but initially represents a +/− protein status (i.e. the protein is either present or absent in the control or leptin-treated sample).

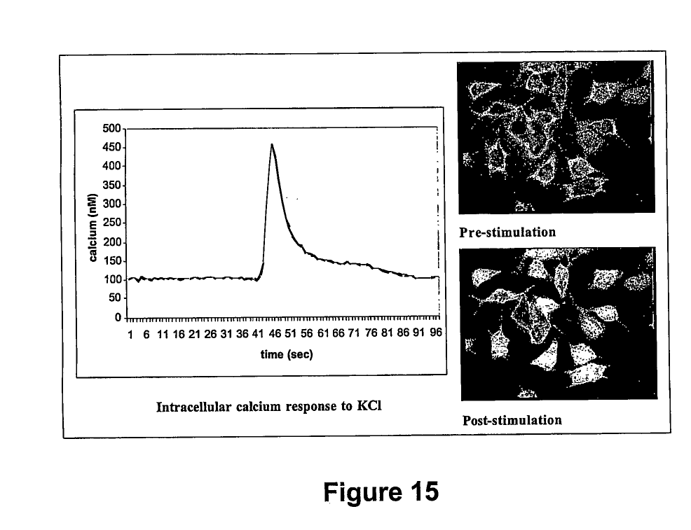

Since many of the peptides and enzyme present in the individual cell lines are secretory products, one representative cell line, N-38 (Also known as NPY-17), was studied for its response to depolarization by addition of 60 mM KCl. FIG. 15 illustrates that the NPY-17 cells exhibit an intracellular calcium response to KCl, indicating that they are neurosecretory cells. NPY-17 neurons were plated in 60 mm plates. Fura-2/AM loaded GT1-7 neurons were continuously monitored by 340/380 ratiometric digital imaging pre (top right panel) and post (lower right panel) stimulation. A representative graph is provided (FIG. 15, left panel) showing the effect of KCl (60 mM) on ([Ca2+]i) as a function of raw 340/380 nM ratios for n=12 cells. KCl (60 mM) caused a significant increase in calcium mobilization in the NPY-17 cells. The representative graph of [Ca2+]i shows a peak value of ˜450 nM. The single spike observed is typical of a functional neurosecretory response.

It has also been found that sequential deletions of the NPY 5′ regulatory region affect differential expression in NPY-17 neurons. NPY-17 cells were transiently transfected with 15 μg of one of the expression vectors containing the human NPY 5′ regulatory region (−1078 bp) and sequential deletion mutants of this same region. Transfections were incubated for 48 hours after the primary transfection of the DNA for 12 hours with the calcium phosphate precipitate. Bars represent the relative LUC activity as compared to vector control activity (pGL2-luc) of each construct. The preliminary data is an average of two independent measurements in duplicate or triplicate.

Such cells can be used to identify modulators of NPY. Such modulators can then subsequently be used to treat NPY-related conditions.

TCAP Responsive Cell Lines

The onset of mood disorders, such as depression or anxiety, involve the altered function of multiple loci in the brain that regulate emotionality, memory and motivation (41-43). However, the inability to describe a cellular and molecular aetiology of these conditions likely reflects, in part, the existence of critical neurological circuits not yet understood. A novel family of neuropeptides on the carboxy-terminus of the teneurin transmembrane proteins (32, 44) is implicated in neuronal communication. In one embodiment, they modulate stress responses and anxiety (data not shown) and likely play a role in some psychiatric illnesses. In another embodiment, they are implicated as modulators of cell cycle and cell proliferation. These sequences are called teneurin C-terminal-associated peptides (TCAP-1 to 4) [SEQ. ID NOs 1-9]. These peptides may also have a cleavable amidation signal at the C-terminus, such as “GRR” or “GKR”. The immortalized cell lines were screened for TCAP responsiveness using rtTCAP-3 [SEQ. ID NO 9] plus amidation signal GKR. TCAP signals through a specific cAMP-dependent G-protein-coupled receptor to modify cell cycle and proliferation in immortalized neurons by regulating genes associated with growth suppression and promotion, and neuronal signaling.

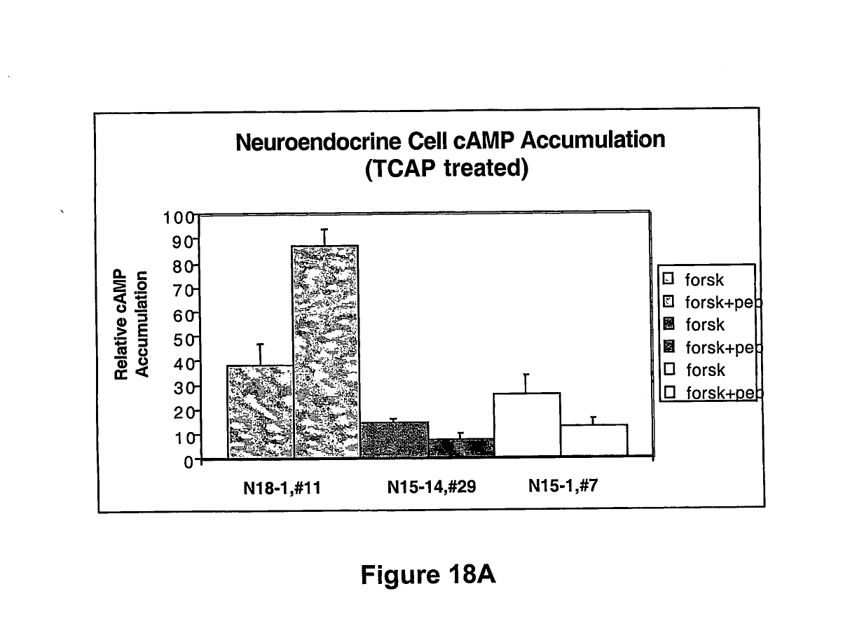

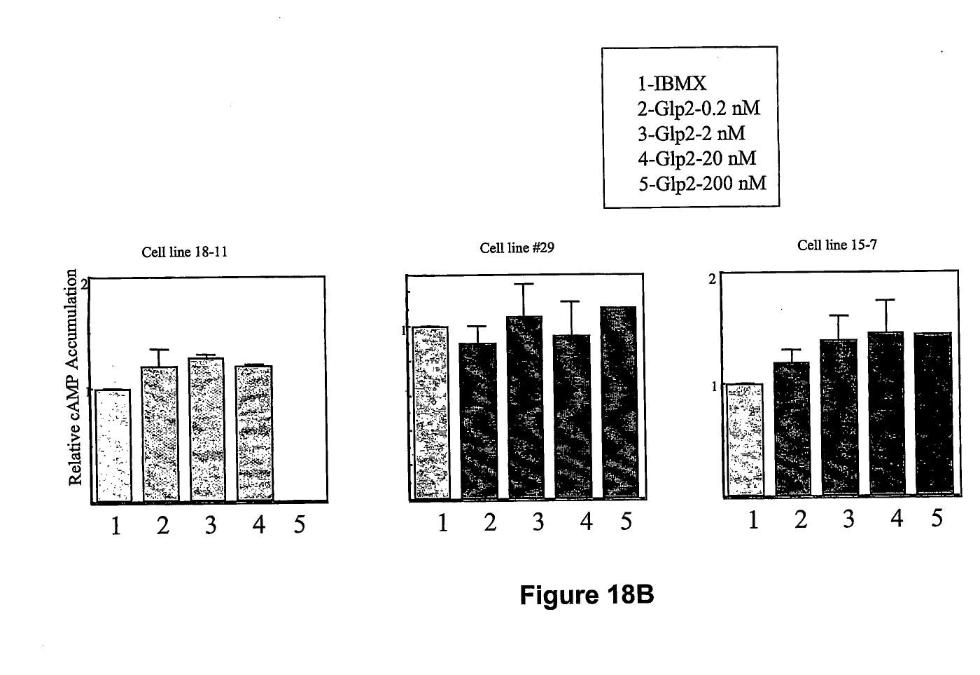

The functional cAMP response of the immortalized subclones to TCAP peptide stimulation was also assessed and the results are shown in FIG. 18. N-7 (N-15-1, #7), N-22 (N-18-1, #11), N-38 and N-29 (N-15-14, #29) have been identified as TCAP responsive cell lines. N-7, N-22 and N-29 were analyzed for the cAMP response to peptide stimulation. The subclones were split into 24 well plates. Cells were starved for 1 hour in DMEM without FBS, then medium was replaced with 0.5 ml fresh DMEM (without FBS) with the compounds as indicated. In FIG. 18A, neurons were exposed to 10⁻⁷ M (100 nM) TCAP peptide (a newly discovered stress-regulating peptide provided by Dr. D. Lovejoy at the University of Toronto), rtTCAP-3 [SEQ ID NO 9]. In FIG. 18B, they were exposed to glucagon-like peptide 2 at 0.2, 2, or 20 nM for 15 minutes. All peptides were diluted in DMEM containing IBMX (100 μM). After a 15 minute incubation at 37° C., 1 ml of ice-cold ethanol was added to each well. Cells were scraped from the plate and kept at −20° C. until the amounts of intracellular cAMP were determined in triplicate by RIA (Biotechnologies Inc., Stoughton, Mass.) according to the manufacturer's instructions.

Further, to demonstrate that the cell lines can be used as a model for studying TCAP responsiveness, modulation, and in screening for TCAP modulators, microarray studies were performed on 1 nM TCAP-1 [SEQ ID NO 5 plus amidation signal GRR at C-terminus] treated N38 hypothalmic cells, which do not possess either CRF receptor subtype (Table 4). RNAs isolated from treated and untreated cells were analyzed on oligonucleotide arrays representing 12,884 mouse genes (Affymetrix, http://www.affymetrix.com). Standard filtering (p<0.005) and hierarchical clustering algorithm (average linage method: GeneSpring software—Silicon Genetics) identified significant changes in the expression of 4, 841/12,885 genes with 166 genes showing 1.5 fold down-regulation and 35 genes up-regulation in the TCAP-1-treated cells compared to the untreated cells. At 16 hours post-treatment, a significant decrease occurred among several genes, notably, GAS5, SDPR and CD95 that have been associated with growth arrest or apoptotic events (45-47). In contrast, upregulated genes including MK167, MOP3 and GDAP10 have been associated with cell proliferation and cell cycle modulation (48-50). A G-protein coupled receptor-related signal transduction pathway is indicated by the regulation of genes, CREM, AKAP8, AKAP95 and PDE6A. Downstream effectors of RAS such as EFK1 and RGL were also down regulated. Downregulation of the A kinase anchoring protein AKAP95 but upregulation of AKAP8 suggests that TCAP may act, in part, by changing the targeting pattern of PKA (51). The upregulation in inducible nitric oxide (INOS), a intracellular voltage-gated chloride channel (CLCN3) and the serotonin transporter (SLC6A4) may reflect the down stream actions of cAMP-mediated signal cascade and indicates the potential for TCAP to be involved in neuronal signaling systems. A role in interneuron communication by TCAP is also indicated by the modulation of genes associated with the regulation of vesicle trafficking. Thus, the TCAP responsive cell lines can be used to screen for modulators of neuronal function that affect growth, differentiation and communication.

While certain cell lines, such as NPY-17, and TCAP responsive cell lines N-7, N-22, N-38 and N-29, have been particularly described, the present invention is not limited to only these cell lines. The invention also provides the cell lines listed and characterized in Table 3.

Applications

Using the immortalization, selection and screening methods of the present invention, many unique immortalized hypothalamic cells, of both neuroendocrine and glial origin, have been isolated, a few of which are indicated in Table 3. The cells generated by the method of the present invention are unique compared to other immortalized cell lines because the cells did not originate as a tumor, but were transformed only by the expression of SV40 large T-antigen in monolayer primary culture. The transformation process therefore is kept to a minimum (it has been postulated that tumorigenesis often results from more than one genetic event), which should be more representative of the primary cell. As such, the present invention provides a method for generating immortalized hypothalamic neuronal cell lines, more preferably derived from embryonic cells. In another embodiment the cell lines are derived from murine cells. Further the cells are considered to be neuron-like due to the expression of neuron-specific enolase, a specific neuronal marker (3). These immortalized, clonal cell lines provide valid model systems for molecular and biochemical investigations on the regulation of specific hormones, characteristics of their respective secretory neuronal population and an unlimited source of homogenous cell material and of the neuropeptide itself. The method of the present invention has been used to isolate a virtually unlimited number of cell models from the hypothalamus by immortalizing hypothalamic primary cell cultures. A number of the immortalized cells have been cloned and characterized.

For instance, the cell lines of the present invention can be used to identify modulators of respective neuropeptides, that may for instance be used in the treatment or regulation of a disorder associated with the said neuropeptide. This can be done by incubating the cells of the cell line with the candidate modulator under conditions that promote neuropeptide expression and then monitoring the effect of the candidate modulator on the neuropeptide. Suitable monitoring techniques are known in the art, such as monitoring neuropeptide expression (e.g. mRNA or peptide level), and/or neuropeptide activity (38, 39) Neuropeptide activity can be monitored by incubating the cell line under conditions that promote neuropeptide expression, in the presence of a known neuropeptide substrate and the candidate modulator and monitoring the effect of the candidate modulator on neuropeptide activity and/or expression. Such effect can be compared to a control level (internal or external controls). For instance the control can be neuropeptide activity/expression in the absence of the candidate modulator, in the presence of a known modulator, and/or in just buffer, etc. The present invention is not limited to such controls. Alternatively, such assays can be performed under conditions that inhibit neuropeptide expression and the effect of the candidate modulator can be assessed as above. Modulation is observed when there is an increase, decrease or homeostasis effect on the neuropeptide expression and/or activity.

The cell lines and related methods and modulator screening assays of the present invention, for instance as noted above, are useful for many applications. A few areas where these cell lines are useful include drug discovery, therapeutic drug testing platforms, phenotypic profiling of individual neuronal cell types, gene expression studies, studies of mechanisms of action of novel neuropeptides and neuronal mechanisms, signal transduction in neurons, and receptor cloning and characterization, neuronal regeneration and cell death. They are also useful in proteomic studies, genome-wide gene expression profiling in selected neuronal cell types, neuron-specific transcription factor analysis, neuron-neuron interactions and communication, brain modelling and studies of ion channel function. The cell lines are also useful for studies in neuronal regeneration and cell death, as well as in animal models of disease. The cell lines can also be useful in identifying diagnostic tests for certain medical conditions. As each immortalized cell line of the present invention corresponds to an actual neuronal cell type, these endogenous cell types can be isolated using endogenous characteristic markers identified on the respective immortalized cell line (38-40). Specific cell lines of the present invention or the endogenous non-immortalized cell types identified and isolated may also be useful in the treatment of certain disorders or diseases.

The cell lines can also be used as a source of neuropeptides which in turn can be used in medical treatment, and in screening for modulators of said peptide and in drug design. One aspect of drug design would be based on structural and chemical characteristics of the neuropeptide. For instance, a number of the neuronal cell models reported herein have the potential to produce major neurotransmitters, as many of the lines expressed tyrosine hydroxylase, a marker of catecholaminergic neurons. Cells expressing tyrosine hydroxylase have the potential to produce dopamine, norepinephrine, and epinephrine, depending upon the complement of downstream catalytic enzymes. Other cell lines expressed tryptophan hydroxylase, the rate-limiting enzyme of serotonin production and an important component of melatonin biosynthesis. Due to the involvement of these neurotransmitters in the development of neurological disorders, such as depression, the use of the cell lines to study their regulation is fundamental for the development of satisfactory treatment and diagnostic options. Cell lines were also generated expressing specific releasing peptides, such as growth hormone-releasing hormone, GnRH, or secreted peptides, such as proglucagon-derived peptides, oxytocin or arginine vasopressin.

The above disclosure generally describes the present invention. A more complete understanding can be obtained by reference to the following specific Examples. These Examples are described solely for purposes of illustration and are not intended to limit the scope of the invention. Changes in form and substitution of equivalents are contemplated as circumstances may suggest or render expedient.

### EXAMPLES

The examples are described for the purposes of illustration and are not intended to limit the scope of the invention.

Methods of tissue culture, protein and peptide biochemistry, molecular biology, histology and immunology referred to but not explicitly described in this disclosure and examples are reported in the scientific literature and are well known to those skilled in the art.

### Example 1

### Primary Cell Culture of e15, 17, and 18 Hypothalamus

Mice (BALB/c females and DC1 males from Charles River) were bred and the appearance of a vaginal plug signified embryonic day 0. An entire litter was harvested at embryonic day 15, 17 or 18. Gravid mice were euthanized by CO₂ inhalation. The uterus was removed immediately and placed in a 100 mm tissue culture dish containing phosphate buffered saline (PBS), with 1 mM EDTA, pH 7.4, at 4° C. The dish was transferred to a dissecting microscope where the fetuses were removed from the uterus. The fetuses were decapitated and the heads transferred to a new culture dish containing primary culture medium (DMEM, 10% heat-inactivated defined FBS, 10% heat-inactivated defined horse serum, 1% penicillin-streptomycin, and 20 mM D-glucose (all from Life Technologies/Gibco)). Osmolarity of the culture medium was adjusted to 320-325 mOsm with glucose. The plate contained enough culture medium to cover the head, and no more than three heads per dish were maintained. Dissection was optimized for speed and accuracy. Two needle-point forceps were used to separate meninges and the hypothalamus was transferred to a sterile 15 ml tube containing 1 ml of culture medium at 4° C. Tissue was dissociated into a single cell suspension by trituration with 2-3 fire-polished Pasteur pipettes of decreasing tip diameter (4-6 strokes per pipette). The cell suspension was split into two 75-80 cm² tissue culture flasks, coated with 100 μg/ml poly-L-lysine (Sigma P-6282), with 8 ml of culture medium. Cells were allowed to attach to the flask for 24 h incubating at 37° C. with 5% CO₂. After 24 h of growth the cells were ready for infection with retrovirus.

### Example 2

### Retroviral Infection of Hypothalamic Primary Cell Cultures

Because extreme caution should be used when handling retroviral cultures harbouring oncogenic immortalization factors, such as simian virus large T-antigen, a Level 3 containment room and tissue culture hood was used for this work. The viral supernatant was prepared at a titre between 10⁵-10⁶ cfu/ml. The producer cells harbouring the oncogene (described below) were co-cultured with NIH3T3 cells (host) at a 1:4 ratio. The viral supernatant was collected and assayed for colony forming units/ml of supernatant. The supernatant was stored at −80° C. until primary cell culture infection. After attachment of primary cells to the culture dish for approximately 24 h, cells were incubated for 1 h, twice successively, with fresh virus-containing medium harvested from confluent culture of ψ2 cells (psitex cells) producing a replication-defective, recombinant murine retrovirus. This virus, constructed by using the pZIPNeo SV(X) 1 vector, harbours the intact cDNA sequence for simian virus (SV40) large T antigen and neomycin resistance gene. Retrovirus infected cells (after 48 h in culture medium with retrovirus) were incubated with medium containing geneticin (G 418) in highly selective concentration (400-600 μg/ml for initial selection, 250 μg/ml for cell maintenance). Geneticin selection of infected cells was performed parallel with geneticin treatment of non-infected cells as a control.

### Example 3

### Analysis of Cell Populations and Subcloning of Cell Lines

Resistant colonies, appearing after 2-3 weeks, were picked using cloning cylinders and further expanded (40). After several passages cells were sub-cloned and during each expansion step, a part of the culture was frozen in cryoprotectant medium and stored in liquid nitrogen. The growth curves of cloned cell lines, still representing a mixed population of hypothalamic cells, displayed a doubling time of approximately 24-48 h. The new clones were selected if they demonstrated a predominantly neuronal lineage morphology, as small, rounded or ovoid perikarya and long neuritic processes. Generally, the cloned lines form monolayers, grow rapidly and retain growth contact inhibition. Further expansion of a cell line was performed only after the evidence for the expression of at least large T antigens neuron-specific enolase and lack of glial fibrillary acidic protein, determined either through reverse transcriptase polymerase chain reaction (RT-PCR) reaction or Northern blotting. A cell line with a mixed population of hypothalamic cells was further subcloned through successive dilutions of the trypsinized cells into 96 well tissue culture plates coated with poly-L-lysine. The optimal dilution allowed only 1 or 2 cells per well. The cells were incubated in conditioned medium, i.e. medium taken from the mixed cultures, at a 1:1 ratio with culture medium, DMEM with 15% FBS (Life Technologies/Gibco). Cell colonies were allowed to grow and then successively split into 24 well plates, and finally 60 mm plates. These clones were grown until the cells could be frozen in liquid nitrogen and also harvested for RNA extraction. The RNA was used to make cDNA, and the clonal cell line was analysed for expression of a large number of markers indicating a unique neuronal phenotype. Immortalized cell lines were grown in DMEM supplemented with 10% FBS, 20 mM glucose and penicillin-streptomycin maintained at 37° C. with 5% CO₂.

### Example 4

### Cell Line Screening

Each cell line was analyzed for the expression of specific markers by reverse transcriptase PCR (RT-PCR). First strand cDNA was synthesized from 10 μg of DNasel-treated RNA, using SuperScript II reverse transcriptase (RT) (Life Technologies/Gibco). The RT reaction was primed with random primers. CDNA synthesis was followed by RNase H (180 U/ml) digestion of RNA in a total volume of 20 μL. Control reactions were performed where amplification was carried out on samples in which the RT was omitted (RT-). Whenever possible, primer sequences flanked an intron, as an extra control for DNA contamination. In most cases, the agarose gels were transferred for Southern blot analysis with an internal primer sequence as a probe. All products were sequenced to confirm identity. Amplification details can be found in Table 2.

### Example 5

### Calcium Imaging

Ca2+ imaging experiments were performed with Fura2 (Molecular Probes) using an Olympus IX70 inverted epifluorescence microscope, an Ultrapix camera with an EEV CCD37-10-1-019 chip, a monochromator, and a PC computer with Merlin imaging software (Life Science Resources) using standard Fura2 optics and imaging techniques. The extracellular solution used for imaging experiments consisted of (in mM): 140 NaCl, 4 KCl, 2 CaCl₂, 1 MgCl₂, 10 glucose and 10 HEPES at pH 7.3. Cells were loaded with 4 μM Fura2-AM for 40 minutes at 37° C. in standard extracellular solution. Intracellular free Ca2+ ([Ca2+]i) concentrations calculated using the Grynkiewicz equation, [Ca2+]i=Kd×□×(R−Rmin)/(Rmax−R) (52). Results are shown in FIG. 15.

### RESULTS

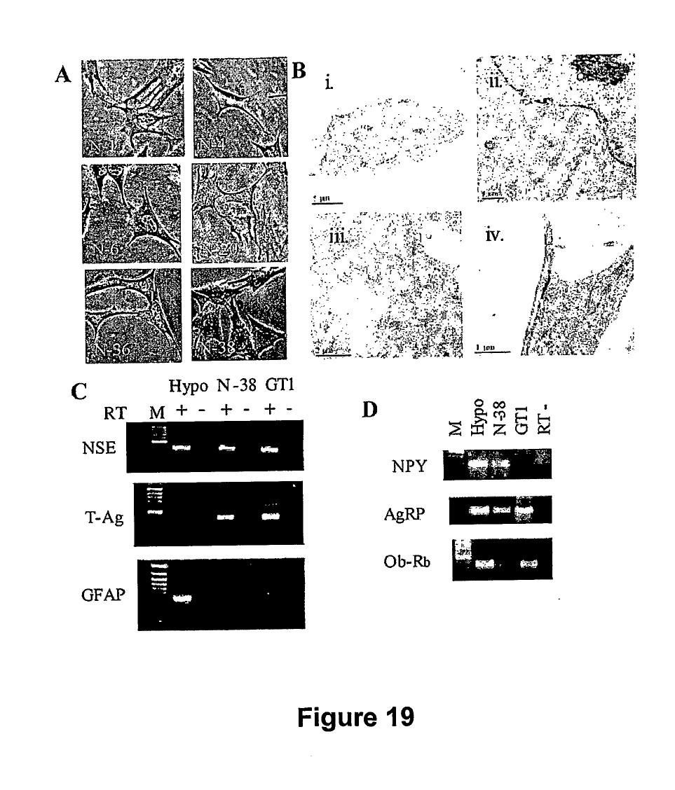

As seen in Table 3, each cell line has a distinct cell morphology, indicating that they represent unique cell types demonstrating the potential diversity from the heterogeneous cells, although all of the cells have some common characteristics, such as overall neuronal phenotype and the appearance of neurites (FIG. 19A). Using electron microscopy it was found that the cells exhibit dense core granules, indicative of secretory neurons, and from cell-cell contacts, with what appears to be the presence of dense material at the contact point, indicative of what has been seen previously at a synaptic cleft (53). (FIG. 19B). The lines are stable and maintain expression of SV40 T-antigen and NSE, but do not express GFAP (FIG. 19B). For instance, the N-38 cell line has been passaged to P-44 and maintains SV40 T-antigen expression after 2 years of continuous growth. Further, all cell lines express syntaxin-1, a SNARE protein complex member localized to the presynaptic plasma membrane that has an indispensable role in neurosecretion (54). These immortalized, clonal cell lines provide valid model systems for molecular and biochemical investigations on the regulation of specific hormones, characteristics of their respective secretory neuronal population and an unlimited source of homogenous cell material and of the neuropeptide itself. The neuropeptides can be used in the treatment of related neurological conditions.

Expression profiles of the hypothalamic cell lines characterized to date are described in Table 3. Known hypothalamic markers were examined with particular attention to neuropeptides linked to energy homeostasis, specific releasing hormones, and enzymes responsible for neurotransmitter synthesis. A wide variety of neuronal phenotypes were generated, often with co-expression of multiple markers, confirming what has been detected in vivo through mainly immunocytochemical methodology (55). The neurons expressing peptides linked to energy homeostasis expressed peptide profiles consistent with those reported by immunochemistry or in situ hybridization (4).

For instance all neuropeptide Y (NPY)-expressing neurons, thought to have orexigenic properties in feeding behaviour, also expressed agouti-related peptide (AgRP), but not proopiomelanocortin (POMC), a precursor to the anorexigenic neuropeptide alpha-melancortin-stimulating hormone (56, 57). The successful generation of cell models from the hypothalamus that were not previously available allows the study of transcriptional regulation of many genes associated with complex neuroendocrine pathways. Specifically, very little is known of the control of melanin-concentrating hormone, urocortin, neurotensin, proglucagon, and growth hormone-releasing hormone in the hypothalamus the cell lines provided by this invention are the first available to study the regulation of these genes. Importantly, many of the lines expressing peptides associated with energy homeostasis also express the long form of the leptin receptor, ObR_{b} and suppressor of cytokine signaling (SOCS), a downstream effector molecule of ObR_{b} (58). As an example, the N-38 cell line expresses NPY, AgRP and ObR_{b} but not POMC (FIG. 19D). Interestingly, although not expected, all of the cell lines epressed AgRP and GnRH, indicating a potential role for these petides in development. Although a number of cell lines expressed POMC, expression of cocaine and amphetimine regulated transcript (CART) was not detected in any of the cell lines, which may suggest a limited window of expression of this peptide during development.

TABLE 1
Cell Line Names

| CELL LINE | COMMON NAME | eDAY |
|---|---|---|
| N-1 | N-15-1, #1 | 15 |
| N-2 | N-15-1, #2 | 15 |
| N-3 | N-15-1, #3 | 15 |
| N-4 | N-15-1, #4 | 15 |
| N-5 | N-15-1, #5 | 15 |
| N-6 | N-15-1, #6 | 15 |
| N-7 | N-15-1, #7 | 15 |
| N-8 | N-15-1, #8 | 15 |
| N-11 | N-15-1, #11 | 15 |
| N-15 | N-15-1, #15 | 15 |
| N-17 | N-15-1, #17 | 15 |
| N-18 | N-15-1, #18 | 15 |
| N-19 | N-18-1, #6 | 18 |
| N-22 | N-18-1, #11 | 18 |
| N-20 | N-15-14, #20 | 15 |
| N-25 | N-15-14, #25 | 15 |
| N-29 | N-15-14, #29 | 15 |
| N-36 | N-15-14, #36 | 15 |
| N-37 | N-17, #2517-6 | 17 |
| N-38 | N-17, #37-1 (NPY-17) | 17 |
| N-39 | N-17, #2517-3 | 17 |
| N-40 | N-17, #1 | 17 |
| N-41 | N-17, #2 | 17 |
| N-42 | N-17, #3 | 17 |
| N-43 | N-17, #4 | 17 |
| N-44 | N-17, #6 | 17 |
| N-45 | N-17, #7 | 17 |
| N-46 | N-17, #8 | 17 |
| N-47 | N-17, #9 | 17 |

TABLE 2
Amplification details used in the RT-PCR analysis of gene expression.

| | | ANNEALING | Amplicon | POSITIVE | CROSS |
|---|---|---|---|---|---|
| GENE | PRIMER SEQUENCE | (35 cycles) | (bp) | CONTROL | INTRON |
| T-Antigen (T-Ag) | S: agaggaatctttgcsgctaa | 60 | 433 | GT 1-7 | yes |
| | AS: ctaaacacagcatgactcaa | | | | |
| Neuron-Specific Enolase (NSE) | S: ctgatgctggagttggatg | 60 | 391 | GT 1-7 | yes |
| | AS: cttcgctgttctccaggatat | | | | |
| Glial Fibrillary Acidic | S: ctgaggctggaggcagagaac | 57 | 621 | Hypothalamus | yes |
| Protein (GFAP) | : AS: cctgtaggtggcgatctcgat | | | | |
| Neuropeptide Y (NPY) | S: taggtaacaagcgaatgggg | 60 | 282 | Hypothalamus | yes |
| | AS: acatggaagggtcttcaagc | | | | |
| | IP: gctctgcgacactacatcaa | | | | |
| Neurotensin (NT) | S: staggaatgaacttcagctg | 60 | 498 | Hypothalamus | yes |
| | AS: gtaggaggccctcttgagtat | | | | |
| Proglucagon (proGlu) | S: ttcaccagcgactacagcaaa | 60 | 351 | Hypothalamus | yes |
| | AS: ggtttgaatcsgccagttgat | | | | |
| | IP: agtgatgtgagttcttacttg | | | | |
| Tyrosine Hydroxylase (TH) | S: cagctggaggatgtgtctca | 60 | 140 | Hypothalamus | yes |
| | AS: ggcatgacggstgtactgtg | | | | |
| | IP: cggtctactgtctgcccgtg | | | | |
| Growth Hormone Releasing | S: ttgtgatcctcatcctcaccag | 60 | 128 | Hypothalamus | yes |
| Hormone | AS: atcactttccgggcatacag | | | | |
| (GHRH) | IP: atgccatcttcaccaccaac | | | | |
| Corticotropin-Releasing | S1: attctgatccgcatgggtgaagaatacttc | 60 | 162 | Hypothalamus | no |
| Hormone (CRH) | AS1: taattaggggtatataggctctctccctg | | | | |
| | IP: tgcagaatcgttttggccaagcgcaacatt | | | | |
| Galanin (Gal) | S: catgccattgacaaccacag | 55 | 320 | Hypothalamus | yes |
| | AS: ggattggcttgaggagttgg | (30 cycles) | | | |
| | IP: atgtgcccctgcctgagagc | | | | |
| Proopiomelanocortin (POMC) | S: tagatgtgtggagctggtgc | 60 | 149 | Hypothalamus | yes |
| | AS: cagtcaggggctgttcatct | | | | |
| | IP: aacctgctggcttgcatccg | | | | |
| Neurofilament protein (NF) | S: cgaagagcgagatggccaggtac | | | | yes |
| | AS: cactctgcaagcaaacagatact | | | | |
| Agouti Related Peptide (AgRP) | S: agggcatcagaaggcctgaccagg | 60 | 252 | Hypothalamus | yes |
| | AS: ttgaagaagcggcagtagcacgt | | | | |
| | IP: atccacagaaccgcgagtct | | | | |
| Cocaine and Amphetamine | S: agctcccgcctgcggctgct | 60 | 299 | Hypothalamus | yes |
| Regulated Transcript (CART) | AS: cagtcacacagcttcccgatcc | | | | |
| | IP: tgttgcagatcgaagcgttg | | | | |
| Urocortin (Ucn) | S: gcgtcttcagcccgtccccggggacagagt | 60 | 455 | Hypothalamus | yes |
| | AS: ccgatcacttgcccaccgaatcgaatatg | | | | |
| | IP: gctacgctcctggtggcgttgctgctcttg | | | | |
| | IP:-intron: tggaactggactggcacagc | | | | |
| Arginine Vasopressin (Avp) | S: gccgcgggcatctgctgcagcgatg | 60 | 223 | Hypothalamus | yes |
| | AS: tcagtagacccggggcttggcagaa | | | | |
| | IP: agcaacgccacacagctggac | | | | |
| Thyrotropin Releasing | S: ggaccttggctgatgatggct | 60 | 592 | Hypothalamus | yes |
| Hormone (TRH) | AS: tcaggcattaagccaccctcc | | | | |
| | IP: aagacgttgaagccgaagag | | | | |
| Suppressor of Cytokine | S: tccacgctggctccgtgcg | 57 | 603 | Hypothalamus | no |
| Signaling (SOCS3) | AS: gctccttaaagtggagcatca | | | | |
| Melanin-concentrating | S: ttaatgctggctttttctttgttt | | | | |
| Hormone (MCH) | AS: accgctctcgtcgtttttgta | 50 | 185 | Hypothalamus | yes |
| | IP: gcttccaagtccataaggaa | | | | |
| Tryptophan Hydroxylase (TPH) | S: cattcctccgaaagagggagagtgact | 60 | 254 | Hypothalamus | |
| | AS: agctgatcgggcgagtccaccgaga | | | | |
| Oxytosin (OXT) | S: ccagtctcgcttgctacctg | 55 | 310 | Hypothalamus | yes |
| | AS: gggctgcagcagatgcctgt | | | | |
| | IP: cgctgcttcggaccaagcat | | | | |
| Gonadotropin-Releasing | S: actgtgtgtttggaaggctgc | 62 | 162 | GT 1-7 | yes |
| Hormone (GnRH) | AS: ttccagagctcctcgcagatc | (30 cycles) | | | |
| | IP: actgtccactggccccgttc | | | | |
| Antrogen Receptor (AR) | S: acgtcctggaagccattgagcc | 60 | 560 | GT1-7 | yes |
| | AS: cttggtgagectggtggaagcgc | | | | |
| Estrogen Receptor α(ERα) | S: gaattcaattctgacaatcgacgccag | 57 | 344 | GT1-7 | yes |
| | AS: gaattcgtgcttcaacattctccctcc | | | | |
| Estrogen Receptor ⊕(ER⊕) | ₁: gaattctagccacccactgccaatcat | 59 | 407/332 | GT1-7 | yes |
| | AS₁: gaattccacacctttctctcctggatg | | | | |
| Leptin Receptor (Ob-Rb) | S: atgacgcagtgtactgctg | 60 | 356 | Hypothalamus | yes |
| | AS: gtggcgagtcaagtgaacct | | | | |
| | IP: tgtgcagctgaggtatcaca | | | | |
| Glucagon-like-peptide 2 | S: ccccaaccaagcccggcagtg | 60 | 187 | Hypothalamus | no |
| Receptor (Glp2R) | AS: tcttacttgcttgatggaaacc | | | | |
| | IP: cctgcggcccaggggcctgag | | | | |
| Aromatase (Arom) | S: ggattggaagtgcctgcaactact | 60 | 400 | Hypothalamus | |
| | AS: gagcatgttagaggtgtccagcat | | | | |
| | IP: cggcatgcatgagaacggca | | | | |
| Orexin (OX) | S: cctgagctccaggcaccatgaact | | | | |
| | AS: tggttaccgttggcctgaaggagg | 60 | 232 | Hypothalamus | yes |
| | IP: cagaagacgtgttcctgccg | | | | |

TABLE 3
Cell Lines Screening

| | | | | N- | | | N- | N- | N- | N- | N- | N- | N- | N- | N- | N- | N- |
|---|---|---|---|---|---|---|---|---|---|---|---|---|---|---|---|---|---|
| MARKER | N-1 | N-2 | N-3 | 4 | N-6 | N-7 | 8 | 11 | 19 | 22 | 20 | 25 | 29 | 36 | 37 | 38 | 39 |
| Tantigen | + | + | + | + | + | + | + | + | + | + | + | + | + | + | + | + | + |
| NSE | + | + | + | + | + | + | + | + | + | + | + | + | + | + | + | + | |
| GFAP | − | − | − | − | − | − | − | − | − | − | − | − | − | − | N/A | − | |
| NT | − | − | − | − | − | − | − | − | − | − | − | − | − | + | − | − | + |
| ERα | + | + | + | + | + | + | + | + | + | + | + | + | + | + | + | + | |
| ERβ | + | − | + | + | + | + | + | + | + | + | + | + | + | + | + | + | |
| Tph | − | − | − | − | − | + | − | − | N/A | + | + | w, | − | + | strong | w | |
| | | | | | | | | | | | | but RT- | | | | | |
| | | | | | | | | | | | | weaker | | | | | |
| Socs-3 | + | − | + | + | − | + | + | + | N/A | + | + | + | + | + | + | + | + |
| AR | − | N/A | + | w | + | − | w | − | N/A | − | N/A | N/A | N/A | N/A | N/A | + | − |
| G2R | − | − | w | − | − | + | − | − | + | + | − | − | + | − | − | − | |
| CRF | − | − | − | − | − | − | − | − | − | − | − | − | − | − | − | − | w |
| GnRH | | + | + | + | + | + | + | + | + | + | + | + | + | + | + | w | |
| POMC | − | − | w | + | − | − | w | + | + | + | + | − | + | − | + | − | |
| Gal | + | − | + | − | − | + | + | w | − | − | w | w | w | + | − | − | − |
| Lep | + | + | w | w | + | − | + | + | + | + | − | − | + | w | w | w | + |
| Receptor | | | | | | | | | | | | | | | | | |
| Agrp | + | + | + | + | + | + | + | + | + | + | + | + | + | + | + | + | + |
| Cart | − | − | − | − | − | − | − | − | − | − | − | − | − | − | − | − | − |
| NPY | w | − | − | − | − | − | − | w | − | − | − | − | + | w | − | + | − |
| proGlu | − | − | − | − | w | − | + | − | − | w | − | − | w | − | + | − | w |
| TH | − | + | w | + | + | + | + | + | + | − | + | w | + | + | + | − | |
| GHRN | w | w | + | − | + | − | w | w | + | + | − | w | + | + | + | + | |
| Avp | + | + | + | + | + | + | + | + | + | + | + | + | w | + | w | w | |
| proTRH | − | − | − | − | − | − | − | − | − | − | − | − | − | − | − | − | − |
| Ucn | + | − | w | w | + | − | − | + | − | − | + | + | − | + | − | − | + |
| MCH | N/A | N/A | + | + | N/A | + | N/A | N/A | N/A | N/A | N/A | N/A | + | w | w | + | + |
| orexin | − | − | − | − | − | − | − | − | N/A | − | − | − | − | − | − | − | − |
| DAT | − | − | − | − | − | strong | − | + | N/A | − | − | w | w | − | − | − | |
| CRFR1 | − | N/A | − | − | bigger | − | − | − | N/A | − | bigger | − | − | − | N/A | − | |
| | | | | | size | | | | | | size | | | | | | |
| CRFR2 | − | N/A | − | − | − | − | − | − | N/A | − | − | − | − | − | N/A | − | |
| Aromatase | + | N/A | − | − | − | − | − | + | N/A | − | − | − | − | N/A | + | strong | |
| GnRH | − | N/A | − | − | − | − | − | − | N/A | − | − | − | − | N/A | − | − | |
| Receptor | | | | | | | | | | | | | | | | | |
| Insulin | + | + | + | + | + | + | + | + | N/A | + | + | + | + | + | + | + | + |
| receptor | | | | | | | | | | | | | | | | | |
| Oxytocin | + | N/A | − | a bit? | w | + | + | + | N/A | + | + | + | + | N/A | + | + | |
| Ten M-1 | − | − | − | − | − | − | − | − | − | − | − | − | − | − | − | − | |
| Ten M-2 | − | − | − | − | − | − | − | − | − | − | − | − | − | − | − | − | |
| Ten M-4 | + | + | + | + | + | − | + | + | + | − | − | − | + | + | + | − | |
| GHS-R | N/A | N/A | N/A | N/A | N/A | N/A | N/A | N/A | N/A | N/A | N/A | N/A | N/A | N/A | N/A | − | + |
| Leptin | | | | | | | | | | | | | | | | | |
| som | | | | | | | | | | | | | | | | | |
| NTR | − | − | N/A | N/A | − | + | − | + | N/A | w | N/A | N/A | N/A | N/A | w | − | w |
| mc3R | | | | | | | | | | | | | | | | | |
| mc4R | N/A | N/A | N/A | N/A | N/A | N/A | N/A | N/A | N/A | N/A | N/A | N/A | N/A | N/A | N/A | − | + |
| NPY-Y1 | | | | | | | | | | | | | | | | | |
| NPY-Y2 | | | | | | | | | | | | | | | | | |
| CRLR | N/A | N/A | N/A | N/A | N/A | N/A | N/A | N/A | N/A | N/A | N/A | N/A | N/A | N/A | N/A | − | + |
| Ghrelin | + | − | N/A | N/A | − | + | + | − | N/A | + | N/A | N/A | N/A | N/A | + | + | + |
| Ghrelin | − | − | N/A | N/A | + | + | + | − | N/A | − | N/A | N/A | N/A | N/A | + | − | + |
| variant | | | | | | | | | | | | | | | | | |

| | N- | N- | N- | N- | N- | N- | N- | N- | N- | | N- | N- | N- | N- | N- | | | | |
|---|---|---|---|---|---|---|---|---|---|---|---|---|---|---|---|---|---|---|---|
| MARKER | 20/1 | 20/2 | 25/2 | 25/3 | 25/4 | 29/1 | 29/2 | 29/3 | 29/4 | N-36/1 | 36/2 | 40 | 41 | 42 | 43 | N-44 | N-45 | N-46 | N-47 |
| Tantigen | + | + | + | + | + | + | + | + | + | + | + | + | + | + | + | + | + | + | + |
| NSE | | | | | | | | | | | | | | | | | | | |
| GFAP | | | | | | | | | | | | | | | | | | | |
| NT | + | + | + | − | − | + | + | + | + | strong | + | + | + | + | + | + | + | + | + |
| ERα | | | | | | | | | | | | | | | | | | | |
| ERβ | | | | | | | | | | | | | | | | | | | |
| Tph | | | | | | | | | | | | | | | | | | | |
| Socs-3 | + | + | + | + | N/A | + | + | + | + | + | + | + | + | + | + | + | + | + | + |
| AR | + | + | w | − | − | w | − | w | − | − | + | + | + | + | + | + | + | + | + |
| G2R | | | | | | | | | | | | | | | | | | | |
| CRF | − | − | − | − | − | − | − | − | − | − | − | − | − | − | − | + | − | − | − |
| GnRH | | | | | | | | | | | | | | | | | | | |
| POMC | | | | | | | | | | | | | | | | | | | |
| Gal | + | − | − | − | − | − | − | − | − | + | − | + | + | + | + | + | + | + | + |
| Lep Receptor | + | + | + | + | − | + | + | + | + | + | + | + | + | + | + | + | w | + | + |
| Agrp | + | + | + | + | + | + | + | + | + | + | + | + | + | + | + | + | + | + | + |
| Cart | − | − | − | − | − | − | − | − | − | − | − | − | − | − | − | + | − | − | − |
| NPY | − | − | + | w | − | w | − | − | + | − | − | + | + | + | + | + | + | + | + |
| proGlu | − | + | − | − | − | − | − | − | − | − | − | + | + | − | − | − | − | − | − |
| TH | | | | | | | | | | | | | | | | | | | |
| GHRH | | | | | | | | | | | | | | | | | | | |
| Avp | | | | | | | | | | | | | | | | | | | |
| proTRH | − | − | − | − | − | − | − | − | − | − | − | − | − | − | − | − | − | − | − |
| Ucn | + | + | + | + | w | + | + | + | + | + | + | + | + | + | + | w | w | + | + |
| MCH | + | + | + | + | w | + | + | + | + | + | + | + | + | + | + | + | + | + | + |
| orexin | − | + | − | − | − | − | − | − | − | − | − | − | − | − | − | − | − | − | − |
| DAT | | | | | | | | | | | | | | | | | | | |
| CRFR1 | | | | | | | | | | | | | | | | | | | |
| CRFR2 | | | | | | | | | | | | | | | | | | | |
| Aromatose | | | | | | | | | | | | | | | | | | | |
| GnRH Receptor | | | | | | | | | | | | | | | | | | | |
| Insulin receptor | + | + | + | + | + | + | + | + | + | + | + | + | + | + | + | + | + | + | + |
| Oxytocin | | | | | | | | | | | | | | | | | | | |
| New-1 | | | | | | | | | | | | | | | | | | | |
| New-2 | | | | | | | | | | | | | | | | | | | |
| New-4 | | | | | | | | | | | | | | | | | | | |
| GHS-R | − | − | − | − | N/A | + | + | + | − | − | − | − | − | − | − | + | − | + | − |
| Leptin | | | | | | | | | | | | | | | | | | | |
| som | | | | | | | | | | | | | | | | | | | |
| NTR | + | w | − | − | N/A | w | − | − | − | + | w | − | − | − | w | − | − | − | − |
| mc3R | | | | | | | | | | | | | | | | | | | |
| mc4R | − | − | + | + | N/A | + | − | − | − | − | + | + | + | + | + | ? | + | + | + |
| NPY-Y1 | | | | | | | | | | | | | | | | | | | |
| NPY-Y2 | | | | | | | | | | | | | | | | | | | |
| CRLR | + | + | − | − | N/A | + | − | − | − | + | + | + | + | + | + | + | + | + | + |
| Ghrelin | + | + | + | − | N/A | + | + | + | + | + | + | + | + | + | + | + | + | + | + |
| Ghrelin variant | − | + | − | + | N/A | − | + | − | + | w | − | + | + | − | − | + | − | + | − |

TABLE 4
Genes Regulated by TCAP-1 at 16 hours

| | | Affimetrix | Acc No. | | Fold |
|---|---|---|---|---|---|
| Cluster | Gene | Probe No. | GB | Function | change |
| Growth/ | GAS5 | 98530 | AI849615 | Growth arrest specific transcript | 0.46 |
| Differentiation | SDPR | 160373 | AI839175 | Serum deprivation response protein | 0.57 |
| | PPAN | 160802 | AA674812 | Peter Pan homologue | 0.62 |
| | CD95 | 102921 | M83649 | Fas antigen | 0.61 |
| | CRD-BP | 102627 | AF061569 | CRD-binding protein | 0.59 |
| | SSG1 | 160298 | AW122012 | Steroid sensitive gene 1 | 0.62 |
| | DLP1/2 | 97353 | AI837497 | DAB2 interacting protein | 0.68 |
| | GBP3 | 103202 | AW047476 | Guanylate binding protein | 0.63 |
| | P202 | 161173 | AV229143 | 202 interferon activatable protein | 0.61 |
| | CAI1 | 103441 | AI94248 | Casein kinase II | 0.61 |
| | INI1B | 99924 | AW121845 | Integrase interacting protein 1B | 0.48 |
| | MMP1 | 100484 | X66473 | Matrix metalloproteinase 1 | 0.55 |
| | MMP10 | 94724 | Y13185 | Matrix metalloproteinase 10 | 0.59 |
| | PTK7 | 92325 | AI326889 | Receptor protein tyrosine kinase | 1.53 |
| | P204 | 98466 | M31419 | Interferon activatable protein | 1.85 |
| | MKI67 | 161931 | AV309347 | Cell cycle protein regulator | 1.70 |
| | MOP3 | 102382 | AB014494 | Circadian rhythm regulator | 1.57 |
| | ST7 | 160591 | AI504013 | Suppressor of tumourigenicity | 1.97 |
| | GDAP10 | 94192 | Y17860 | Ganglioside induced diff. protein 10 | 1.62 |
| Signalling/ | ERK1 | 101834 | ZI4249 | Mitogen activated protein kinase | 0.64 |
| Communication | ALK3 | 92767 | DI6250 | Bone morphogenic protein receptor | 0.60 |
| | BMP4 | 93456 | L47480 | Bone morphogenic protein-4 | 0.52 |
| | IL1R | 93914 | M20658 | Interleukin 1 receptor | 0.60 |
| | GR | 98818 | X04435 | Glucocorticoid receptor | 0.66 |
| | BARK1 | 104270 | AA982714 | β adrenorgic receptor kinase 1 | 0.61 |
| | CAMIII | 92631 | M19380 | Calmodulin III | 0.53 |
| | PCDHγ | 160976 | AA222943 | protocadherin γ | 0.42 |
| | AKAP95 | 95001 | AB028920 | A kinase anchor protein 95 | 0.60 |
| | TTF-1IP | 161019 | W41560 | TTF-1 interacting peptide | 0.50 |
| | CREMβ1 | 100533 | M60285 | cAMP-responsive element modulator | 1.61 |
| | AKAP8 | 161088 | AV171460 | A kinase anchor protein 8 | 1.58 |
| | PDE6A | 100696 | X60664 | cGMP Phosphodiesterase α | 1.68 |
| | INOS | 104420 | U43428 | Inducible nitric oxide synthetase | 1.50 |
| | FNBX | 92754 | D49920 | Ferredoxin-NADP reductase | 1.61 |
| | SLC6A4 | 161695 | AV230927 | Serotonin transporter | 1.53 |
| | CLCN3 | 94465 | AF029347 | Chloride channel protein 3 | 1.66 |
| Processing | ARF1 | 95156 | AI1853873 | ADP ribosylation factor 1 | 0.63 |
| | CLM2-B | 93492 | AB013469 | Cytohesin-2 | 0.63 |
| | YIPID | 99675 | AI839766 | Rab-mediated membrane transport | 1.88 |
| | RAB10 | 160149 | AI841543 | Ras oncogene homologue | 1.62 |
| | GP25L2 | 100074 | AW046723 | gp25L brings cargo forward from ER | 1.53 |
| | AP4S1 | 104561 | AI847561 | Adaptor related protein complex | 1.52 |

The change in expression levels is indicated relative to the untreated control cell for the same time period of 16 hours. Values>1.5 fold or <0.70 fold were considered significant.

### REFERENCES

1. Everitt, B. J. & Hokfelt, T. 1990 Neuronendocrine anatomy of the hypothalamus. Acta. Neurochir. Suppl. (Wien.) 47: 1-15
2. Hoffman, G. E., Smith, M. S. & Verbalis, J. G. 1993 c-Fos and related immediate early gene products as markers of activity in neuroendocrine systems. Front. Neuroendocrinol. 14: 173-213
3. Duforny, L., Warembourg, M. & Jolivet, 1999 Quantitative studies of progesterone receptor and nitric oxide synthase colocalization with somatostatin, or neurotensin, or substance P in neurons of the guinea pig ventorlateral hypothalamic nucleus: an immunocytochemical triple-label analysis. J. Chem. Neuroanat. 17: 33-43
4. Elmquist, J. K., Elias, C. F. & Saper, C. B. 1999 From lesions to leptin: hypothalamic control of food intake and body weight. Neuron 22: 221-232
5. Smith, M. S. & Grove, K. L. 2002 Integration of the regulation of reproductive function and energy balance: Lactation as a model. Front. Neuroendrocrinol 23: 225-256
6. McKay R D G 1989 The origins of cellular diversity in the mammalian central nervous system. Cell 58: 815-821
7. Cepko C L 1989 Immortalization of neural cells via retrovirus-mediated oncogene transduction. Ann. Rev. Neurosci. 12: 47-65
8. de Vitry F, Camier M, Czernichow P, Benda P, Cohen P, Tixier-Vidal A 1974 Establishment of a clone of mouse hypothalamic neurosecretory cells synthesizing neurophysin and vasopressin. Proc. Natl. Acad. Sci. USA 71: 3575-3579
9. Rosenfeld M G, Crenshaw Ed, Lira S A, Swanson L, Borrelli E, Heyman R, Evans R M 1988 Transgenic mice: applications to the study of the nervous system. Ann Rev Neurosci 11: 353-372
10. Hanahan D 1989 Transgenic mice as probes into complex systems. Science 246: 1265-1275
11. Windle J J, Weiner R I, Mellon P L 1990 Cell lines of the pituitary gonadotrope lineage derived by targeted oncogenesis in transgenic mice. Mol. Endocrinol. 4: 597-603
12. Hanahan D 1985 Heritable formation of pancreatic b-cell tumours in transgenic mice expressing recombinant insulin/simian virus 40 oncogenes. Nature 315: 115-122
13. Gardette, R., Courtois, M. & Bisconte, J. C. 1982 Prenatal development of mouse central nervous structures: time of neuron origin and gradients of neuronal production. A radioautographic study. J Hirnforsch 23: 415-431.
14. Brown M et. al. 1986 A recombinant murine retrovirus for simian virus 40 large T cDNA transforms mouse fibroblasts to anchorage-independent growth. J. Virol. 60: 290-293.
15. Ornitz D M, Hammer R E, Messing A, Palmiter R D, Brinster R L 1987 Pancreatic neoplasia induced by SV40 T-antigen expression in acinar cells of transgenic mice. Science 238: 188-193
16. Efrat S, Linde S, Kofod H, Spector D, Delannoy M, Grant S, Hanahan D, Baekkeskov S 1988 β-cell lines derived from transgenic mice expressing a hybrid insulin gene-oncogene. Proc. Natl. Acad. Sci. USA 85: 9037-9041
17. Mellon P L, Windle J J, Weiner R 1991 Immortalization of neuroendocrine cells by targeted oncogenesis. Rec. Prog. Horm. Res. 47: 69-96
18. Mellon P L, Windle J J, Goldsmith P, Pedula C, Roberts J, Weiner R I 1990 Immortalization of hypothalamic GnRH neurons by genetically targeted tumorigenesis. Neuron 5:1-10
19. Shkreta L, Belsham D D 2000 Establishment of immortalized hypothalamic neuronal cell lines expressing specific neuropeptide genes. Endocrine Society Annual Meeting 82: 284
20. Wahlestedt C, Heilig M 1994 Neuropeptide Y and related peptides. In: Bloom F (ed) Psychopharmacology—the fourth generation of progress, on-line edition. Lippincott Williams & Wilkins, Philadelphia, Pa.: www.acnp.org/g4/GN401000052/CH052.html
21. Stanley B G, Kyrkouli S, Lampert S, Leibowitz S 1986 Neuropeptide Y chronically injected into the hypothalamus: a powerful neurochemical inducer of hyperphagia and obesity. Peptides 7: 1189-1192
22. Terasawa E 2001 Luteinizing hormone-releasing hormone (LHRH) neurons: mechanism of pulsatile LHRH release. Vitam Horm 63:91-129
23. Terasawa E 1995 Control of luteinizing hormone-releasing hormone pulse generation in nonhuman primates. Cell Mol Neurobiol 15:141-164
24. Allen J M, Balbi D 1993 Structure and expression of the neuropeptide Y gene. In: Colmers W, Wahlestedt C (eds) The biology of neuropeptide Y and related peptides. Humana Press, Totowa, N.J.: 43-64
25. Minth C A, Dixon J E 1990 Regulation of the human neuropeptide Y gene. Ann NY Acad Sci 611: 99-110
26. Liu J, Kahn A I, Heikkila P, Voutilainen R 1999 Regulation of neuropeptide Y mRNA expression in cultured human pheochromocytoma cells. Eur J Endocrinol 141: 431-435
27. Higuchi H, Yang H Y, Sabol S L 1988 Rat neuropeptide Y precursor gene expression. mRNA structure, tissue distribution, and regulation by glucocorticoids, cyclic AMP, and phorbol ester. J Bioi Chem 263: 6288-6295
28. Sahu A, Kalra S P, Crowley W R, Kalra P S 1988 Evidence that NPY-containing neurons in the brainstem project into selected hypothalamic nuclei: implication in feeding behavior. Brain Res 9: 376-378
29. Sahu A, Kalra P S, Crowley W R, Kalra S P 1990 Functional heterogeneity in neuropeptide-Y-producing cells in the rat brain as revealed by testosterone action. Endocrinology 127: 2307-2312
30. Kalra P S, Kalra S P 2000 Use of antisense oligodeoxynucleotides to study the physiological functions of neuropeptide Y. Methods 22: 249-254
31. Lerchen R A, Yum D Y, Krajcik R, Minth-Worby C A 1995 Transcriptional vs. posttranscriptional control of neuropeptide Y gene expression. Endocrinology 136: 833-841
32. Oohasi, T. et al., 1999 Mouse Ten-m/Odz is a new family of dimeric type II transmembrane proteins expressed in many tissues. J. Cell Biol. 145: 563-577
33. Rubin, B. P., Tucker, R. P. Martin, D & Chiquet-Ehrismann, R. 1999 Teneurins: A novel family of neuronal cell surface proteins in vertebrates, homologous to the Drosophila pair-rule gene product Ten-m Dev. Biol. 216:195-209
34. Baumgartner, S., Martin, D., Hagios, C. & Chiquet-Ehrismann, R. 1994 Tenm, a Drosophila gene related to tenascin, is a new pair-rule gene. EMBO J. 13:.3728-3740
35. Levin, A., et al., 1994 Odd Oz: a novel Drosophila pair rule gene. Cell 77: 587-598
36. Levine, A. et al., 1997 The genetics and molecular structure of the Drosophila pair-rule gene odd Oz (odz). Gene 200: 59-74
37. Wang, X.-Z. et al., 1998 Identification of novel stress induced genes downstream of chop. EMBO J. 17: 3619-3630
38. Ausubel, F. M. et al., 1988 Current Protocols In Molecular Biology, New York, John Wiley and Sons Inc.
39. Phillip, T. M., Dickens, B. F., 2000 Affinity and Immunoaffinity Purification Techniques, Natick, Mass.: BioTechiques Books
40. Maniatis, T., et al. 1982 Molecular Cloning: A laboratory manual, Cold Spring Harbor Laboratory, New York.
41. Manji, H. K., Drevets, W. C. & Charney, D. S. 2001 The cellular neurobiology of depression. Nature Med. 7: 541-547
42. Drevets, W. C. 2001 Neuroimaging and neuropathological studies of depression: Implications for the cognitive emotional features of mood disorders. Curr. Opin. Neurobiol. 11: 240-249
43. Nestler, E. J. et al. 2002 Neurobiology of Depression. Neuron 34: 13-25
44. Minet, A. D., Rubin, B. P., Tucker, R. P., Baumgartner, S. & Chiquet-Ehrismann, R. 1999 Teneurin-1, a vertebrate homologue of the Drosophila pair-rule gene, Ten-m, is a neuronal protein with a novel type of heparin-binding domain. J. Cell Sci. 112: 2019-2032
45. Coccia, E. M. et al. 1992 Regulation and expression of a growth arrest-specific gene (gas5) during growth differentiation and development. Mol. Cell Biol. 12: 3514-3521
46. Gustincich, S. et al. 1999 The human serum response gene (SDPR) maps to 2q32-q33 and codes for a phosphatidylserine-binding protein. Genomics 57: 120-129
47. Stanger, B. Z. 1996 Looking beneath, the surface: the cell death pathway of Fas/Apo-1 (CD95). Mol. Med. 2: 7-20
48. Kaaijk, P. et al. 2003 Cell proliferation is related to in vitro drug resistance in childhood acute leukaemia. Br. J. Cancer 88: 775-781
49. Liu, H. et al. 1999 Isolation of 10 differentially expressed cDNAs in differentiated Neuro2a cells induced through controlled expression of the CD3 synthetase gene. J. Neurochem. 72: 1781-1790
50. Bunger et al. 2000 Mop3 is an essential component of the master circadian pacemaker in mammals. Cell 103: 1009-1017
51. Fayos, R. et al. 2003 Induction of flexibility through protein-protein interactions, J. Biol. Chem., in press
52. Grynkiewisc, G. et. al., 1985 J. Biol. Chem. 260: 3440-3450
53. Tixier et al.1979 Intl. Rev. Cytol. 58: 291-331
54. Rettig et. al., 2002 Science 298: 781-785
55. Sawchenko et al. 1992 Ciba Found Symp, 168:16-30
56. Broberger, C. et. al. 1998 J. Comp. Neurol. 402: 460-474
57. Hahn, T. M., et; al. 1998 Nat. Neurosci 1: 271-272
58. Bjorbaek, C. et. al., 1999 J. Biol. Chem. 274: 30059-30065

We claim:

1. An immortalized cell line of murine hypothalmic neuronal cells comprising a gene encoding polyoma virus large T antigen operably linked to a promoter and expressing a marker selected from the group consisting of neuropeptide Y, gonadotropin-releasing hormone, growth-hormone releasing hormone (GHRH), TenM 1, 2, 3, 4, arginine vasopressin (AVP), thyrotropin-releasing hormone (TRH), SOCS-3, urocortin, melanocortin-concentrating hormone (MCH), orexin, dopamine transporter, corticotrophin-releasing factor (CRF), gonadotropin releasing hormone receptor, tryptophan hydroxylase, tyrosine hydroxylase, galanin, proopiomelanocortin (POMC), proglucagon, neurotensin, somatostatin, agouti-related protein, cocaine and amphetamine-regulated transcript (CART), leptin, oxytocin, corticotrophin-releasing factor receptor 1 and 2, aromatase, ghrelin, growth hormone secratogue receptor, androgen receptor, estrogen receptor α, estrogen receptor β, leptin receptor, melanocortin-concentrating hormone receptor 3 and 4, neuropeptide Y receptor Y1, neuropeptide Y receptor Y2, calcitonin receptor like receptor, glucagon-like peptide 1 receptor, glucagon-like peptide 2 receptor (Glp-2 receptor), and neurotensin receptor.

2. A mixed cell population comprising the immortalized cell line of claim 1.

3. The immortalized cell line of claim 1 wherein the marker is Glp-2 receptor.

4. The immortalized cell line of claim 1 wherein the marker is neurotensin.

5. The immortalized cell line of claim 1 wherein the marker is proopiomelanocortin (POMC).

6. The immortalized cell line of claim 1 wherein the marker is neuropeptide Y (NPY).

7. The immortalized cell line of claim 1 wherein the marker is proglucagon.

8. The immortalized cell line of claim 1 wherein the marker is growth-hormone releasing hormone.

9. The immortalized cell line of claim 1 wherein the marker is urocortin.

10. The immortalized cell line of claim 1 wherein the marker is melanocortin-concentrating hormone.

11. The immortalized cell line of claim 1 wherein the marker is TenM 4.

12. The immortalized cell line of claim 1 wherein the marker is growth hormone secratogue receptor.

13. The immortalized cell line of claim 1 wherein the marker is ghrelin.

14. An immortalized cell line of claim 1 prepared by the method comprising:
(i) preparing a culture of embryonic hypothalamic cells;
(ii) infecting said culture with a retrovirus encoding a viral oncogene, operably linked to a promoter and a selectable marker;
(iii) isolating transfected cells from non-transfected cells to obtain a culture of immortalized hypothalamic cells;
(iv) subcloning said immortalized cells into sub-cloned populations;
(v) screening said subcloned populations for expression of specific neuronal markers; and
(vi) selecting and further cloning a specific population.

15. A method of obtaining a neuropeptide comprising, culturing the cell line of claim 1 that is known to express said neuropeptide and isolating the expressed neuropeptide.

16. A method for identifying a modulator of a neuropeptide comprising:
(i) providing a cell line as defined in claim 1;
(ii) incubating the cell line in the presence of the candidate modulator; and
(iii) determining the biological effect of said candidate modulator,
wherein said candidate is a modulator if it modulates the neuropeptide expression and/or activity.

17. The method of claim 15 wherein said effect of said candidate modulator can be determined by one of the following methods:
(a) monitoring effects on neuropeptide expression;
(b) incubating the said cell line with a substrate of a neuropeptide and monitoring the effect on substrate metabolites;
(c) binding assays; or
(d) proteomic profiling in the presence and absence of the said candidate modulator.

18. An immortalized cell line of murine hypothalmic neuronal cells that is responsive to a teneurin C-terminal-associated peptide [TCAP].

19. The cell line of claim 16 wherein the teneurin C-terminal-associated peptide is selected from the group consisting of SEQ ID NOs 1-9.

20. The cell line of claim 16 wherein the teneurin C-terminal-associated peptide is murine TCAP-1 or TCAP-3.

21. The cell line of claim 16 wherein the cell line is selected from the group consisting of N-7, N-22, N-29 and N-38.
